(19) United States
(12) Patent Application Publication
(10) Pub. No.: US 2025/0333512 A1
(43) Pub. Date: Oct. 30, 2025

(54) PLANT-DERIVED THERAPEUTIC MONOCLONAL ANTIBODIES AND THEIR USE IN PREGNANT WOMEN

(71) Applicant: PlantVax, Inc., Rockville, MD (US)

(72) Inventor: Yvonne J. Rosenberg, Washington, DC (US)

(73) Assignee: PlantVax, Inc., Rockville, MD (US)

(21) Appl. No.: 18/698,718

(22) PCT Filed: Oct. 4, 2022
(86) PCT No.: PCT/US22/77493

§ 371 Date: Apr. 4, 2024

Related U.S. Application Data
(60) Provisional application No. 63/252,256, filed on Oct. 5, 2021.

(51) Int. Cl. C07K 16/28; A61K 39/00

(52) U.S. Cl. CPC C07K 16/283; A61K 2039/505; C07K 2317/13; C07K 2317/41; C07K 2317/73; C07K 2317/92

(57) ABSTRACT

Disclosed herein are methods of producing a therapeutic plant antibody or fragment thereof, which is produced in a plant or plant cell to provide a plant antibody or fragment thereof that is unable to substantially cross the placenta if administered to a pregnant mother, but that if produced in a mammal or mammalian cell may cross the placenta and potentially result in damage to a foetus or newborn if administered to a pregnant mother, and so is suitable for use in pregnant subjects. The therapeutic plant antibody can also be used in compositions and medicaments for treating various conditions.

This disclosure relates to therapeutic antibodies, and methods for producing same, that are suitable for use in pregnant subjects. In one particular application, the disclosure may be applied to a therapeutic antibody contraindicated for use in a pregnant subject, to generate a plant form of the antibody with a reduced or no propensity to cause pathology or other deleterious effect in the developing foetus.

### DESCRIPTION OF THE RELATED ART

Monoclonal antibodies (mAbs) have become one of the most important and successful types of therapeutics, revolutionising the treatment of cancer, particularly lymphoid malignancies, and inflammatory diseases such as autoimmune diseases. Indeed, of the top 10 best-selling drugs of 2019, six are mAbs (Informa Pharma Intelligence: www.pharmaintelligence.informa.com). However, many of these hugely successful drugs are contraindicated for pregnancy or are otherwise only recommended for use in pregnant women where “the potential benefits to the mother outweigh the potential risks to the unborn child”. For example, the anti-CD20 mAb rituximab (which is sold under the brand name Rituxan amongst others) is just one example among the present blockbuster therapeutic antibody drugs that is contraindicated for pregnancy. In particular, rituximab carries a US Food and Drug Administration Pregnancy category C (i.e., “Animal reproduction studies have shown an adverse effect on the fetus and there are no adequate and well-controlled studies in humans, but potential benefits may warrant use of the drug in pregnant women despite potential risks.”), and it is recommended that rituximab only “be used in early pregnancy for exceptional cases” and “should be avoided in the last trimester” (D Jacob, MedicineNet: www.medicinenet.com).

It is during the last trimester that the majority of the maternal passive immunity is conveyed to the foetus by its mother, but while this process provides the maternal immunoglobulin G (IgG) antibodies to desirably protect the foetus and newborn against infections from various pathogens (especially viruses and bacteria such as those responsible for measles, rubella, and tetanus), it is considered that, equally, the process may lead to the transfer, undesirably, of other antibodies in the mother such as autoantibodies, therapeutic mAbs and other antibodies which may be regarded as being pathogenic inasmuch as they may impair foetal development, resulting in either transient or permanent damage to the developing foetus. Specific examples of medical conditions caused in the foetus by such pathogenic maternal antibodies include foetal and neonatal alloimmune thrombocytopenia (FNAIT), a severe bleeding disorder in which maternal antibodies cross the placenta and sensitize foetal platelets leading to their elimination, and haemolytic disease of the foetus and newborn (HDFN) in which maternal antibodies sensitize red blood cells (RBCs) in the foetal circulation and subsequently induce lysis that causes foetal anemia.

It is now generally accepted that neonatal Fc receptors (FcγRn) present on the surfaces of placental cells (particularly syncytiotrophoblasts; Simister N E et al., Eur J Immunol 26(7): 1527-1531, 1996) are pivotal for the transfer of maternal antibodies (MatAbs) to the foetus. However, in addition to FcγRn, it is also thought that other IgG-binding Fc receptors, namely the activating Fc gamma receptor types FcγRIIA and FcγRIIIA, may also be required to bind to the MatAbs for placental transfer and/or are responsible for mediating the effector functions of the MatAbs in the foetus (Langel S N et al., PLoS Pathog 16(3): e1008303, 2020). Thus, pathogenic maternal antibodies, which have either been produced by the mother (e.g. autoantibodies) or administered to the mother to treat a disease or condition (e.g. therapeutic mAbs to treat, for example, a cancer), can be delivered via the placenta to the foetus where they can potentially cause a range of deleterious effects such as autoimmunity or the targeting and elimination of cells. For example, therapeutic mAbs that have been proven to be effective against a cancer in the mother may result in injury in the foetus by a number of different effector functions such as neutralising activity, apoptosis-inducing effects or mechanisms involving “killer” cells (e.g. antibody-dependent cellular cytotoxicity (ADCC), complement-dependent cytotoxicity (CDC), and antibody-dependent cellular phagocytosis (ADCP)).

Accordingly, there is a need to develop strategies to regulate the transfer of pathogenic MatAbs such as therapeutic mAbs from a pregnant mother to the foetus by, for example, developing new therapeutic mAbs or versions of therapeutic mAbs that are safe for use during pregnancy (and particularly during the third trimester) because they are either unable to cross the placenta or may otherwise lack the ability to do harm in the foetus.

### SUMMARY OF THE DISCLOSURE

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject.

As described herein is a method of treating a pregnant female in need thereof, wherein said pregnant female is administered a plant antibody or fragment thereof; wherein the plant antibody or fragment thereof is produced in a plant or plant cell to provide a plant antibody or fragment thereof that is unable to substantially cross the placenta if administered to a pregnant mother. In certain embodiments, the plant antibody or antibody fragment thereof is unable to substantially bind to FcγRI, FcγRIIA, FcγRIIB, FcγRIIIA or other Fc receptors, and even more preferably, the plant antibody or antibody fragment thereof is able to substantially bind to FcγRn. In certain embodiments, the plant antibody when produced in a mammal or mammalian cell may cross the placenta and/or is able to substantially bind to FcγRI, FcγRIIA, FcγRIIB, FcγRIIIA or other Fc receptors.

In other aspects, the plant antibody or fragment thereof comprises at least one plant specific glycan, such as, for example, a plant specific glycan comprising at least one sugar selected from the group consisting of: a fucose with a α1-3 linkage and a xylose with a β1,2 linkage. Additionally, the plant specific glycan may not comprise galactose.

More specifically, the plant antibody or fragment thereof has a glycopeptide profile comprising at least one glycan selected from the group consisting of:
GlcNAc₂ Fuc Man₃ Xyl;
GlcNAc₂ Man₃ GlcNAc;
GlcNAc₂ Fuc Man₃ Xyl GlcNAc;
GlcNAc₂ Man₃ Xyl GlcNAc;
GlcNAc₂ Fuc Man₃ Xyl GlcNAc₂; and
GlcNAc₂ Fuc Man₃ Xyl GlcNAc₂ Hex.

Additionally, the glycopeptide profile comprises at least one glycan of a relative percentage selected from the group consisting of:
at least 2% of GlcNAc₂ Fuc Man₃ Xyl;
at least 2% of GlcNAc₂ Man₃ GlcNAc;
at least 2% of GlcNAc₂ Fuc Man₃ Xyl GlcNAc;
at least 2% of GlcNAc₂ Man₃ Xyl GlcNAc;
at least 2% of GlcNAc₂ Fuc Man₃ Xyl GlcNAc₂; and at least 0.5% GlcNAc₂ Fuc Man₃ Xyl GlcNAc₂ Hex.

In even further embodiments, the glycopeptide profile of the antibody comprises at least one glycan of a relative percentage selected from the group consisting of:
2-10% of GlcNAc₂ Fuc Man₃ Xyl;
2-20% of GlcNAc₂ Man₃ GlcNAc;
2-30% of GlcNAc₂ Fuc Man₃ Xyl GlcNAc;
10-40% of GlcNAc₂ Man₃ Xyl GlcNAc;
20-60% of GlcNAc₂ Fuc Man₃ Xyl GlcNAc₂; and
0.5-6% GlcNAc₂ Fuc Man₃ Xyl GlcNAc₂ Hex.

Additionally, the glycan is selected from:
GlcNAc₂ Fuc Man₃ Xyl as shown in Table 4;
GlcNAc₂ Man₃ GlcNAc as shown in Table 4;
GlcNAc₂ Fuc Man₃ Xyl GlcNAc as shown in Table 4;
GlcNAc₂ Man₃ Xyl GlcNAc as shown in Table 4;
GlcNAc₂ Fuc Man₃ Xyl GlcNAc₂ as shown in Table 4; and
GlcNAc₂ Fuc Man₃ Xyl GlcNAc₂ Hex as shown in Table 4.

In additional embodiments, the plant antibody is selected from: a) a human Fab and a human Fc; b) a humanised Fab and a humanized Fcs from another animal; c) a plant antibody or fragment thereof comprising a tag to target cellular compartments which modify glycans; d) the plant antibody or fragment thereof of c), wherein the tag is a KDEL tag; and/or e) an IgG1, IgG2, IgG3 or IgG4 isotype.

In certain aspects, the plant antibody, if produced in a mammal or mammalian cell, is capable of causing damage to a foetus or newborn if administered to a pregnant mother.

In additional embodiments, the plant or plant cell is selected from: a) is a member of the Solanaceae family; b) a tobacco plant; c) a member of the Nicotiana genus; d) Nicotiana benthamiana; or e) Nicotiana tabacum; f) a plant cell transiently expressing the antibody; g) a plant comprising a polynucleotide integrated into the genome, wherein said polynucleotide encodes the antibody.

Additionally, the plant antibody is substantially non-oxidised. In preferred embodiments, the substantially non-oxidised plant antibody or fragment thereof comprises at least 95% methionine and 5% or less methionine sulfoxide. In further embodiments, the plant antibody or fragment thereof is selected from any one of the antibodies listed in Table 1 or Table 2.

Plant antibodies as described herein can be used to treat a pregnant female suffering from cancer, infectious disease, or an autoimmune disease.

In preferred embodiments, the cancer or condition is selected from the group consisting of an adrenal tumor, an acinar sarcoma, a Astrocytoma, a bladder cancer, a bone cancer, a brain spinal cord cancer, a metastatic brain tumor, a breast cancer, a carotid body tumor, a cervical cancer, a chondrosarcoma, a spinal tumor, a kidney Chronic cell carcinoma, a clear cell carcinoma, a colon cancer, a colorectal cancer, fesmoplastic small round cell tumor, a fibroproliferative small round cell tumor, an extra-bone Myxoid chondrosarcoma, a fibrosarcoma, a fibrous dysplasia, a gallbladder or cholangiocarcinoma, a pregnancy trophoblastic disease, a germ cell tumor, a neck cancer, a hepatocellular carcinoma, an islet cell Tumor, Kaposi's sarcoma, a kidney cancer, a leukemia, a liposarcoma/malignant fat Adenoma, a liver cancer, a lymphoma, a lung cancer, a stromal cell tumor, a melanoma, a meningioma, a multiple endocrine tumor, a multiple myeloma, a myelodysplastic syndrome, a nerve A blastoma, a neuroendocrine tumor, an ovarian cancer, a pancreatic cancer, a papillary thyroid cancer, a parathyroid tumor, a peripheral schwannomas, a pituitary tumor, a prostate cancer, a posterior uveal melanoma, a primary central nervous system tumora renal metastasis cancer, a rhabdoid tumor, a rhabdomyosarcoma, a sarcoma, a skin cancer, a soft tissue sarcoma, a squamous cell carcinoma, a stomach cancer, a synovial sarcoma, a testicular cancer, a thymic carcinoma, a thymoma, a metastatic thyroid cancer, and a uterine cancer.

In further embodiments, the autoimmune disease or condition is selected from the group consisting of: type I diabetes mellitus (T1D), Crohn's disease, ulcerative colitis, myasthenia gravis, vitiligo, Graves' disease, Hashimoto's disease, Addison's disease, autoimmune gastritis, autoimmune hepatitis, rheumatoid disease, systemic lupus erythematosus, progressive systemic sclerosis and variants, polymyositis, dermatomyositis, primary biliary cirrhosis, autoimmune thrombocytopenia, Sjogren's syndrome, multiple sclerosis and psoriasis.

It is to be understood that both the foregoing general description and the following detailed description are exemplary and explanatory only and are intended to provide further explanation of the disclosure as claimed. The accompanying drawings are included to provide a further understanding of the disclosure and are incorporated in and constitute part of this specification, illustrate several embodiments of the disclosure, and together with the description serve to explain the principles of the disclosure.

### DESCRIPTION OF THE DRAWINGS

Embodiments are illustrated by way of example (and not limitation) in the figures of the accompanying drawings, in which like references, indicate similar elements and in which:

FIG. 1 shows percent mAb levels in six mother-baby pairs at birth. Mothers were infused with one, two or three mAb (see Table 3). The antibodies are shown on the X-axis and were compared to the levels (ug/mL) of the infused antibodies presented as a percentage on the Y-axis. While the YTE and LS mutants engineered to increase affinity for FcRn (see FIG. 5) very slightly improved transfer, only mammalian mAbs were significantly transferred across the placenta. Nb: Nicotiana benthamiana. Mammalian (Mam) mAbs include PGT121 produced in CHO cells and 830A produced in HEK293 cells.

FIG. 2A shows the pharmacokinetics of Nb PGT121-YTE mutant in mother #1 and her baby #1.1. Mother #1 was infused with 5 mg/kg of plant PGT121-YTE. Black down arrows show times of PGT121-YTE injection and the red upward arrow indicates the day of birth. Mother #1 received two injections to maintain high levels of the infused antibody.

FIG. 2B shows the pharmacokinetics of Nb PGT121-YTE mutant in mother #2 and her baby #2.1. Mother #2 was infused with 5 mg/kg of plant PGT121-YTE. Black down arrows show times of PGT121-YTE injection and red upward arrows indicate day of birth. Mother #2 received two injections to maintain high levels of the infused antibody.

FIG. 2C shows the pharmacokinetics of Nb WT PGTG121 in mother #3 and her baby #3.1. Mother #3 was infused with 5 mg/kg of plant WT PGTG121. The black down arrow shows the time of WT PGTG121 injection and the red upward arrow indicates day of birth.

FIG. 2D shows the pharmacokinetics of Nb WT PGTG121 in mother #4 and her baby #4.1. Mother #4 was infused with 5 mg/kg of plant WT PGTG121. Black down arrows show times of WT PGTG121 injection and the red upward arrow indicates day of birth. Mother #4 received two injections to maintain high levels of the infused antibody.

FIG. 3 shows placental transfer of mAbs to babies from a macaque injected with three different antibodies, Nb VRC07-523-LS, and mammalian PGT121 and 830A. Mother #6 was injected with 5 mg/kg of each of the three different mAbs 6 days prior to birth. Mother #6 and baby #6.1 were assayed for the presence of Nb VRC07-523-LS, and mammalian PGT121 and 830A. The levels in the babies are depicted in darker shades than the corresponding mAbs levels in the mother. Only the mammalian PGT121 and 830A mAbs crossed the placenta.

FIG. 4 compares plant and mammalian PGT121 binding to macaque FcRgRIIa, FcRgIIb and FcgRIII using biolayer interferometry (BLI), receptor on solid phase.

FIG. 5A compares SPR response curves and equilibrium binding constants (K_{D}) of plant- and mammalian-derived PGT121 to human and macaque FcRn. mAbs were directly printed onto the SPR chip and analyzed for binding to human (A) over the range of 1,000-15.6 nM. Wild type antibodies included plant-derived PGT121 and VRC07-523, and mammalian (293F)-derived PGT121 and 830A and Herceptin.

FIG. 5B compares SPR response curves and equilibrium binding constants (K_{D}) of plant and mammalian PGT121 to human and macaque FcRn. mAbs were directly printed onto the SPR chip and analyzed for binding to rhesus macaque (B) over the range of 1,000-15.6 nM. Wild type antibodies included plant-derived PGT121 and VRC07-523, and mammalian-derived PGT121 (CHO) and 830A (HEK293) and Herceptin.

FIG. 5C shows the comparison of the equilibrium binding constants (K_{D}) measured by SPR of plant-derived PGT121 and the PGT121 YTE and PGT121 LS Fc mutants to macaque FcRn confirming the higher binding of the engineered YTE and LS mutants. Wild type antibodies included plant-derived PGT121 and VRC07-523, and mammalian-derived PGT121 (CHO) and 830A (HEK293) and Herceptin. As shown in FIG. 5A-C plant PGT121 bound to both human and macaque FcRn, with better binding to the macaque FcRn than the mammalian PGT121.

FIG. 6 shows the comparison of ability of plant and mammalian PGT121 to induce FcR effector function as measured using an antibody-mediated phagocytosis (ADCP) assay. (Chan et al. J Immunol. 197(7):2936-47 (2016)). Plant PGT121 was less efficient in this functional phagocytosis assay than mammalian PGT121 reflecting reduced binding to FcRs.

FIG. 7 shows antibody binding to FcγRI (CD64), which was measured using an IL-2 ELISA. The antibodies tested were plant produced Nb PGT121-LS, Nb PGT121-YTE, Nb PGT121, Nb PGT121-KDEL and Nb VRC01-KDEL, and mammalian PGT121, VRC07-523, VRC07-523-LS, 830A, with a LS HIG positive control. Both plant and mammalian antibodies bound FcγRI. The results confirm that plant mAbs bind and trigger IL-2 production in BW cells transfected with FcγRI, although at a slower rates than the mammalian counterparts.

FIG. 8 shows antibody binding to FcγRIIA (CD₃₂A), which was measured using an IL-2 ELISA. The antibodies tested were the same as those in FIG. 7. The results show that mammalian and control antibodies but not plant antibodies bound FcγRIIA and trigger IL-2 production in BW cells transfected with FcγRIIA.

FIG. 9 shows antibody binding to FcγRIIB (CD₃₂B), which was measured using an IL-2 ELISA. The antibodies tested were the same as those in FIG. 7. Only mammalian antibodies bound FcγRIIB.

FIG. 10 shows antibody binding to FcγRIII (CD16), which was measured using an IL-2 ELISA. The antibodies tested were the same as those in FIG. 7. All plant produced antibodies except Nb PGT121-KDEL did not bind FcγRIII (CD16), whilst the mammalian antibodies, positive control and Nb PGT121-KDEL did bind. Addition of the ER-retrieval KDEL tag, resulted in a Nb PGT121-KDEL mAb lacking the complex glycans fucose or xylose and recovery of significant binding to FcγRIIIA and IL-2 production.

FIG. 11 shows the no FcγR negative control, which was measured using an IL-2 ELISA. The antibodies tested were the same as those in FIG. 7. No antibodies bound.

FIG. 12 shows the deconvoluted full MS spectrum of glycopeptide ³⁰⁸EEQYN*STYR³¹⁶ for mammalian PGT121 and glycoforms corresponding to each peak. The asparagine site NST on N-312 carried the N-glycosylation motif NST and was found to be fully glycosylated (99.98%). The analysis identified 17 glycoforms of which 96.48% were fucosylated. For FIG. 12, N-312 is equivalent to N297 due to the difference in the length of the CDRs in the Fab region.

FIG. 13 shows the deconvoluted full MS spectrum of glycopeptide ³⁰⁸EEQYN*STYR³¹⁶ for mammalian 830A and glycoforms corresponding to each peak. The asparagine site NST on N-328 carried the N-glycosylation motif NST and was found to be fully glycosylated (99.99%). The analysis identified 12 glycoforms of which 98.65% were fucosylated. For FIG. 13, N-328 is equivalent to N297 due to the difference in the length of the CDRs in the Fab region.

FIG. 14 shows the deconvoluted full MS spectrum of glycopeptide ³⁰⁸EEQYN*STYR³¹⁶ mammalian VRC07-523 and glycoforms corresponding to each peak. The asparagine site NST on N-327 carried the N-glycosylation motif NST and was found to be fully glycosylated (99.98%). The analysis identified 14 glycoforms of which 98.71% were fucosylated. For FIG. 14, N-327 is equivalent to N297 due to the difference in the length of the CDRs in the Fab region.

FIG. 15 shows the deconvoluted Full MS spectrum of glycopeptide ³⁰⁸EEQYN*STYR³¹⁶ for mammalian VRC07-523-LS and glycoforms corresponding to each peak. The asparagine site NST on N-327 carried the N-glycosylation motif NST and was found to be fully glycosylated (99.98%). The analysis identified 13 glycoforms of which 92.34% were fucosylated. For FIG. 15, N-327 is equivalent to N297 due to the difference in the length of the CDRs in the Fab region.

FIG. 16 shows the deconvoluted full MS spectrum of glycopeptide ³⁰⁸EEQYN*STYR³¹⁶ for plant Nb PGT121-LS and glycoforms corresponding to each peak. The asparagine site NST on N-312 carried the N-glycosylation motif NST and was present in both glycosylated (71.5%) and non-glycosylated forms (28.5%). The analysis identified 15 glycoforms of which 61.99% were fucosylated. For FIG. 16, N-312 is equivalent to N297 due to the difference in the length of the CDRs in the Fab region.

FIG. 17 shows the deconvoluted full MS spectrum of glycopeptide ³⁰⁸EEQYN*STYR³¹⁶ for plant Nb VRC07-523-LS and glycoforms corresponding to each peak. The asparagine site NST on N-312 carried the N-glycosylation motif NST and was in both glycosylated (73.95%) and non-glycosylated forms (26.05%). The analysis identified 15 glycoform of which 58.49% were fucosylated. For FIG. 17, N-312 is equivalent to N297 due to the difference in the length of the CDRs in the Fab region.

### DETAILED DESCRIPTION OF THE DISCLOSURE

Before describing the present disclosure in detail, it is to be understood that this disclosure is not limited to particularly exemplified materials or process parameters as such may, of course, vary. It is also to be understood that the terminology used herein is for the purpose of describing particular embodiments of the disclosure only, and is not intended to be limiting of the use of alternative terminology to describe the present disclosure.

All publications, patents and patent applications cited herein, whether supra or infra, are hereby incorporated by reference in their entirety for all purposes. The reference to any prior art in this specification is not, and should not be taken as, an acknowledgement or any form of suggestion that such prior art forms part of the common general knowledge.

In some cases, a single embodiment may, for succinctness and/or to assist in understanding the scope of the disclosure, combine multiple features. It is to be understood that in such a case, these multiple features may be provided separately (in separate embodiments), or in any other suitable combination. Alternatively, where separate features are described in separate embodiments, these separate features may be combined into a single embodiment unless otherwise stated or implied. This also applies to the claims which can be recombined in any combination. That is a claim may be amended to include a feature defined in any other claim. Further a phrase referring to “at least one of” a list of items refers to any combination of those items, including single members. As an example, “at least one of: a, b, or c” is intended to cover: a, b, c, a-b, a-c, b-c, and a-b-c.

As used in this specification and the appended claims, the singular forms “a,” “an” and “the” include plural nouns unless the content clearly dictates otherwise. For example, reference to “a polypeptide” includes a mixture of two or more such polypeptide molecules or a plurality of such polypeptide molecules. Similarly, reference to a “polynucleotide” includes a mixture of two or more such polynucleotide molecules or a plurality of such polynucleotide molecules.

As used herein, the term “comprise” or variations thereof such as “comprises” or “comprising” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers, unless otherwise stated or implied. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed disclosure. As used herein, the term “consisting” is used to indicate the presence of the recited integer (e.g., a feature, element, characteristic, property, method/process step or limitation) or group of integers (e.g., features, elements, characteristics, properties, method/process steps or limitations) alone.

The disclosure will be described in more detail below.

### A. Definitions

In the present disclosure, a “polynucleotide” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The terms “percent (%) sequence similarity”, “percent (%) sequence identity”, and the like, generally refer to the degree of identity or correspondence between different nucleotide sequences of nucleic acid molecules or amino acid sequences of polypeptides that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wisconsin), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990, 87:2264, modified as in Karlin and Altschul, Proc. Natl. Acad. Sci. USA 1993, 90:5873-5877. Such an algorithm is incorporated into the NBLAST and XBLAST programs of Altschul et al, J. Mol. Biol. 1990; 215: 403. BLAST nucleotide searches can be performed with the NBLAST program, score=100, wordlength=12, to obtain nucleotide sequences homologous to sequences of the disclosure. BLAST protein searches can be performed with the XBLAST program, score=50, wordlength=3, to obtain amino acid sequences homologous to protein sequences of the disclosure. To obtain gapped alignments for comparison purposes, Gapped BLAST can be utilized as described in Altschul et al, Nucleic Acids Res. 1997, 25:3389. Alternatively, PSI-Blast can be used to perform an iterated search that detects distant relationship between molecules. See Altschul et al., (1997) supra. When utilizing BLAST, Gapped BLAST, and PSI-Blast programs, the default parameters of the respective programs (e.g., XBLAST and NBLAST) can be used. See ncbi.nlm.nih.gov/BLAST/on the World Wide Web.

Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In a preferred embodiment, the percent identity between two amino acid sequences is determined using the algorithm of Needleman and Wunsch (J. Mol. Biol. 1970, 48:444-453), which has been incorporated into the GAP program in the GCG software package (Accelrys, Burlington, MA; available at accelrys.com on the World Wide Web), using either a Blossum 62 matrix or a PAM250 matrix, a gap weight of 16, 14, 12, 10, 8, 6, or 4, and a length weight of 1, 2, 3, 4, 5, or 6. In yet another preferred embodiment, the percent identity between two nucleotide sequences is determined using the GAP program in the GCG software package using a NWSgapdna.CMP matrix, a gap weight of 40, 50, 60, 70, or 80, and a length weight of 1, 2, 3, 4, 5, or 6. A particularly preferred set of parameters (and the one that can be used if the practitioner is uncertain about what parameters should be applied to determine if a molecule is a sequence identity or homology limitation of the disclosure) is using a Blossum 62 scoring matrix with a gap open penalty of 12, a gap extend penalty of 4, and a frameshift gap penalty of 5.

The term “pharmaceutically acceptable” is used to refer to a non-toxic material that is compatible with a biological system such as a cell, cell culture, tissue, or organism. Examples of pharmaceutically acceptable excipients, carriers, buffers, and/or diluents are familiar to one of ordinary skill in the art and can be found, e.g. in Remington's Pharmaceutical Sciences (latest edition), Mack Publishing Company, Easton, Pa. For example, pharmaceutically acceptable excipients include, but are not limited to, wetting or emulsifying agents, pH buffering substances, binders, stabilizers, preservatives, bulking agents, adsorbents, disinfectants, detergents, sugar alcohols, gelling or viscosity enhancing additives, flavoring agents, and colors. Pharmaceutically acceptable carriers include macromolecules such as proteins, polysaccharides, polylactic acids, polyglycolic acids, polymeric amino acids, amino acid copolymers, trehalose, lipid aggregates (such as oil droplets or liposomes), and inactive virus particles. Pharmaceutically acceptable diluents include, but are not limited to, water and saline.

As used herein, “FcgR”, “FcyR” and “FcR” are used interchangeably and are intended to refer to the same molecule. For example, “FcgRIIA”, “FcγRIIa” and “FcRIIA” refer to the same molecule.

### B. Description of the Disclosure

The present disclosure arises from research in primates working to protect foetuses against HIV infection from the mother. The research unexpectedly found that plant produced monoclonal antibodies do not cross the placenta in primates and unlike rodents, that the neonatal Fc receptor (FcRN) is not sufficient for an antibody to cross the placenta, and it is believed that particular glycans play a role in regulating whether or not an antibody crosses the placenta. This contrasts with mammalian produced antibodies which are able to cross the placenta. As such, the use of particular glycans may provide versions of therapeutic mAbs that are safe for use during pregnancy (and particularly during the third trimester) because they are substantially unable to cross the placenta.

Many therapeutic antibodies are contraindicated for use during pregnancy. Some examples include rituximab, trastuzumab and cetuximab. Other examples are shown in Table 1 and Table 2 (below). Other examples include therapeutic antibodies for treating infectious diseases, such as Zika and influenza.

Rituximab is an anti-CD20 antibody or fragment thereof approved for the treatment of non-Hodgkins lymphoma (NHL). CD20 is expressed in B-cell malignancy and is also a pan-B cell marker. The adverse effects of anti-CD20 antibody or fragment thereof therapies are clinically tolerable in adults because host damage is limited in B-cell dependent immune-reaction. However, rituximab has been found to cause adverse developmental outcomes including B-cell lymphocytopenia in infants exposed in utero. Rituximab has also been detected postnatally in the serum of infants exposed in utero. As such, the advice is that women should not become pregnant whilst on rituximab or for 12 months after treatment.

The anti-HER2 antibody or fragment thereof trastuzumab, a humanized antibody or fragment thereof, is approved for the treatment of metastatic breast cancer. The main MOAs are thought to be a neutralizing effect and ADCC. Before the clinical induction of trastuzumab, HER2-positive breast cancer exhibited significantly lower prognosis than HER-2-negative breast cancer. However, trastuzumab treatment improved the prognosis of patients with HER-2-positive tumors to the same level as patients with HER-2-negative tumors. However, when given to pregnant women, trastuzumab has caused oligohydramnios and oligohydramnios sequence, manifesting as pulmonary hypoplasia, skeletal abnormalities, and neonatal death.

The anti-EGFR antibody or fragment thereof cetuximab is approved for the treatment of metastatic colorectal cancer. Cetuximab binds EGFR to reduce the growth and viability of tumour cells. An increased incidence of abortion in a dose-dependent manner has been observed in pregnant women being treated with cetuximab.

TABLE 1
THERAPEUTIC ANTIBODIES FOR CANCER
Antibody information obtained from Yasunaga, M, Seminars in Cancer Biology, 64: 1-12 (2020).

| Name | Target | Molecular format | Payload | Indication |
|---|---|---|---|---|
| Rituximab | CD20 | Chimeric IgG1 | | Malignant lymphoma, |
| | | | | Chronic lymphocytic |
| | | | | leukemia |
| Trastuzumab | HER2 | Humanized IgG1 | | Breast/Gastric cancer |
| Gemtuzumab | CD33 | Humanized IgG4 | Calicheamicin | Acutemyeloid leukemia |
| ozogamicin | | | | |
| Alemtuzumab | CD52 | Humanized IgG1 | | Chronic lymphocytic |
| | | | | leukemia |
| Ibritumomab | CD20 | Murine IgG1 | ⁹⁰Y/¹¹¹In | Malignant lymphoma |
| tiuxetan | | | | |
| Tositumomab | CD20 | Murine IgG2a | ¹³¹I | Malignant lymphoma |
| Bevacizumab | VEGFA | Humanized IgG | | Brain tumor, |
| | | | | Lung/Colon/Breast/Cervical/ |
| | | | | Renal cancer |
| Cetuximab | EGFR | Chimeric IgG1 | | Head and neck/Colon |
| | | | | cancer |
| Panitumumab | EGFR | Human IgG2 | | Colon cancer |
| Ofatumumab | CD20 | Human IgG1 | | Chronic lymphocytic |
| | | | | leukemia |
| Catumaxomab | EpCAM/CD3 | MsIgG2a/RatIgG2b | | Malignant ascites with |
| | | | | EpCAM-positive carcinomas |
| Brentuximab | CD30 | Chimeric IgG1 | MMAE | Malignant lymphoma |
| vedotin | | | | |
| Denosumab | RANKL | Human IgG2 | | Bonemetastases |
| Ipilimumab | CTLA4 | Human IgG1 | | Melanoma |
| Pertuzumab | HER2 | Humanized IgG1 | | Breast cancer |
| Obinutuzumab | CD20 | Humanized IgG1 | | Chronic lymphocytic |
| | | | | leukemia |
| Trastuzumab | HER2 | Humanized IgG1 | DM1 | Breast cancer |
| emtansine | | | | |
| Ramucirumab | VEGFR2 | Human IgG1 | | Gastric cancer |
| Siltuximab | IL-6 | Chimeric IgG1 | | Castleman's disease |
| Blinatumomab | CD19/CD3 | scFv/scFv | | Acute lymphocytic leukemia |
| Pembrolizumab | PD-1 | Humanized IgG4 | | Hodgkin's lymphoma |
| Nivolumab | PD-1 | Human IgG4 | | Melanoma, NSCLC |
| Dinutuximab | GD2 | Chimeric IgG1 | | Neuroblastoma |
| Necitumumab | EGFR | Human IgG1 | | |
| Daratumumab | CD38 | Human IgG1 | | Multiplemyeloma |
| Atezolizumab | PD-L1 | Humanized IgG1 | | Bladder cancer |
| Olaratumab | PDGFRa | Human IgG1 | | Soft tissue sarcoma |
| Avelumab | PD-L1 | HumanIgG1 | | Merkel cell carcinoma |
| Durvalumab | PD-L1 | HumanIgG1 | | Bladder cancer |
| Inotuzumab | CD22 | Humanized IgG4 | Calicheamicin | Acute lymphoblastic |
| ozogamicin | | | | leukemia |
| Elotuzumab | SLAMF7 | Humanized IgG1 | | Multiplemyeloma |

TABLE 2
THERAPEUTIC ANTIBODIES FOR AUTOIMMUNE DISEASE
Yasunaga, M, Seminars in Cancer Biology, 64: 1-12 (2020).

| Name | Target | Molecular format | Indication |
|---|---|---|---|
| Muromonab- | CD3 | Murine IgG2a | Transplantation rejection |
| CD3 | | | |
| Daclizumab | CD25 | Humanized IgG1 | Reversal of transplantation rejection |
| Basiliximab | CD25 | Chimeric IgG1 | Reversal of transplantation rejection |
| Inflixima | bTNFα | Chimeric IgG1 | Crohn's disease/Ulcerative |
| | | | colitis/Rheumatoid arthritis/Ankylosing |
| | | | spondylitis/Psoriatic arthritis/Plaque |
| | | | psoriasis |
| Etanercept | TNFα | TNFR2 with Fc | Rheumatoid arthritis/Psoriatic |
| | | portion of human | arthritis/Plaque psoriasis/Ankylosing |
| | | IgG1 | spondylitis |
| Adalimumab | TNFα | Human IgG1 | Rheumatoid arthritis/Juvenile idiopathic |
| | | | arthritis/Psoriatic arthritis/Ankylosing |
| | | | spondylitis/Crohn's disease/Plaque |
| | | | psoriasis |
| Efalizumab | CD11a | Humanized IgG1 | Psoriasis |
| Omalizumab | IgE | Humanized IgG1 | Asthma |
| Natalizumab | VLA-4 | Humanized IgG4 | Multiple sclerosis (relapsing)/Crohn's |
| | | | disease |
| Ranibizumab | VEGF-A | Humanized IgG1 | Neovascular (wet) age-relatedmacular |
| | | Fab fragment | degeneration/Macular edema following |
| | | | retinal vein occlusion |
| Eculizumab | Complement | Humanized IgG2/4 | Paroxysmal nocturnal hemoglobinuria |
| | C5 | | |
| Certolizumab | TNFa | Humanized IgG Fab | Chron's disease/Rheumatoid arthritis |
| pegol | | fragment | |
| Ustekinumab | IL-12/IL-23 | Human IgG1 | Plaque psoriasis |
| Golimumab | TNFa | Human IgG1 | Rheumatoid arthritis/Psoriatic |
| | | | arthritis/Ankylosing spondylitis |
| Canakinumab | IL-1β | Human IgG1 | Cryopyrin-associated periodic |
| | | | syndrome/Tumor necrosis factor receptor |
| | | | associatedperiodic |
| | | | syndrome/Hyperimmunoglobulin D |
| | | | Syndrome/Mevalonate kinase |
| | | | deficiencyFamilial mediterranean fever |
| Tocilizumab | IL-6R | Humanized IgG1 | Rheumatoid arthritis |
| Denosumab | RANKL | Human IgG2 | Osteoporosis |
| Belimumab | BLyS | Human IgG1 | Systemic lupus erythematosus |
| Alemtuzumab | CD52 | Humanized IgG1 | Multiple sclerosis |
| Vedolizumab | Integrin- | Humanized IgG1 | Ulcerative colitis/Crohn's disease |
| | α4β7 | | |
| Mepolizumab | IL-5 | Humanized IgG1 | Asthma |
| Secukinumab | IL-17a | Human IgG1 | Psoriasis/Psoriatic arthritis/Ankylosing |
| | | | spondylitis |
| Reslizumab | IL-5 | Humanized IgG4 | Asthma |
| Brodalumab | IL-17a | Human IgG2 | Psoriasis |
| Ocrelizumab | CD20 | Humanized IgG1 | Multiple sclerosis |
| Dupilumab | IL-4Rα | Human IgG4 | Asthma/Dermatitis |

As would be appreciated by the person skilled in the art, many of these are contraindicated for use during pregnancy, as they can cause harm to the developing foetus or newborn, or inadequate controlled data exists. However, the use of particular glycans on these plant antibodies means that they may be safe for use during pregnancy (and particularly during the third trimester) because they are substantially unable to cross the placenta to do harm in the foetus. By creating the antibodies as described herein, therapeutic antibodies can be generated which are safe to treat females while pregnant, such as, for example, to treat infectious diseases, such as Zika.

As such, in a first aspect there is provided a method of producing a therapeutic plant antibody or fragment thereof; wherein the plant antibody or fragment thereof is produced in a plant or plant cell to provide a plant antibody or fragment thereof that is unable to substantially cross the placenta if administered to a pregnant mother, wherein the antibody if produced in a mammal or mammalian cell may cross the placenta and cause damage to a foetus or newborn if administered to a pregnant mother, comprising: introducing one or more polynucleotide molecule encoding the plant antibody or fragment thereof into the plant or plant cell; growing the plant or plant cell under conditions to express the polynucleotide molecule(s) and to produce the plant antibody or fragment thereof; and isolating the plant antibody or fragment thereof produced by the plant or plant cell.

The term “antibody or fragment thereof” may encompass a single antibody or fragment thereof, or more than one, such as two, three or four different antibodies or fragments thereof. The term “fragment thereof” may encompass diabodies or fragments of an antibody that contain both the Fab and Fc regions.

In certain embodiments, the antibody comprises a human Fab and a human Fc. In alternative embodiments, wherein the antibody comprises a mouse Fab and a human Fc. In particular embodiments, the antibody comprises a humanised Fab and a Fcs from another animal. In certain embodiments, the antibody comprises a tag to target cellular compartments which modify glycans. In some embodiments, the tag is a KDEL tag, which may provide for retention in the ER and production of only OMT glycans and no complex glycans. Other tags that could be used include, but is not limited to ACTS tags to target the antibody to chloroplasts and VTS tag which would target the antibody to vacuoles. In certain embodiments, the antibody belongs to an IgG1, IgG2, IgG3 or IgG4 isotype.

In certain embodiments, polynucleotide molecule(s) may encode a polypeptide of either a heavy chain or a light chain or both a heavy chain and a light chain of a therapeutic antibody or fragment thereof. The antibody or fragment thereof may be selected by the person skilled in the art in view of the particular disease or condition to be treated in the pregnant mother, for example, from the Tables of antibodies disclosed herein, or using searches of journal article databases, antibody databases (eg Biocompare or Antibodypedia), accessdata.fda.gov or other databases, such as the National Center for Biotechnology Information. The sequence of the heavy and light chains of the antibody or fragment thereof may be readily identified by the person skilled in the art using standard keyword or BLAST searches of online databases, such as the National Center for Biotechnology Information, Biocompare or Antibodypedia.

In certain embodiments, the polynucleotide molecule(s) is transiently expressed in the plant or plant cell. As such, this negates the need for producing stable transformants. In alternative embodiments, the polynucleotide molecule(s) is integrated into the genome of the plant or plant cell to provide a transformed plant cell. In other embodiments, the polynucleotide molecule(s) is transiently expressed in the plant or plant cell which is already transformed with the polynucleotide molecule(s). This may further increase expression or production of the antibody or fragment thereof. In particular embodiments, the polynucleotide molecule(s) is codon optimized for the particular expression system. Methods of transiently transfecting plants or plant cells and methods of stably transforming plants or plant cells, are well known in the art. In certain embodiments, transient expression in plants or plant cells relies on either a non-Agrobacterium-mediated or an Agrobacterium-mediated gene delivery approach as is known in the art. In other embodiments, transient expression in plants or plant cells include systems based on plant viruses to deliver the genetic information and combinations (e.g., Magnifection) as is also well known in the art. In certain embodiments, stable transformation relies on either a non-Agrobacterium-mediated or an Agrobacterium-mediated gene delivery approach as is known in the art. In certain embodiments, a plant cell suspension culture is used. Further discussion is provided below in “C. Method of producing the plant antibody or fragment thereof.” Systems and methods of antibody expression in plants would be known to the person skilled in the art and from publications such as Rosenberg et al. PLoS One 8:e58724 (2013).

In certain embodiments, the method further comprises regenerating a transformed plant from a transformed plant cell. Methods of regenerating a transformed plant from a transformed plant cell are well known in the art, involving timed application of auxin(s) and/or cytokinin(s) to induce shoots and roots.

In certain embodiments, the plant or plant cell is a member of the Solanaceae family. In certain embodiments, the plant or plant cell is a tobacco plant. In certain embodiments, the plant or plant cell is a member of the Nicotiana genus. In certain embodiments, the plant or plant cell is Nicotiana benthamiana or Nicotiana tabacum.

In certain embodiments, the plant antibody or fragment thereof is unable to substantially bind to one or more Fc receptors selected from the group consisting of: FcγRI, FcγRIIA, FcγRIIB, FcγRIIIA and other Fc receptors. In certain embodiments, the plant antibody or fragment thereof is unable to substantially bind to FcγRI. In certain embodiments, the plant antibody or fragment thereof is unable to substantially bind to FcγRIIA. In certain embodiments, the plant antibody or fragment thereof is unable to substantially bind to FcγRIIB. In certain embodiments, the plant antibody or fragment thereof is unable to substantially bind to FcγRIIIA. In certain embodiments, the plant antibody or fragment thereof is able to substantially bind to FcγRn. In any of these embodiments, the substantial inability to bind to one or more of FcγRI, FcγRIIA, FcγRIIB, FcγRIIIA or other Fc receptors means that the plant antibody or fragment thereof is unable to substantially cross the placenta. In certain embodiments, the term “is unable to substantially cross the placenta” means that less than 10% of the plant antibody or fragment thereof crosses the placenta to enter foetal circulation, relative to an equivalent mammalian produced therapeutic antibody or fragment thereof. In certain embodiments, less than 9%, less than 8%, less than 7%, less than 6%, less than 5%, less than 4%, less than 3%, less than 2% or less than 1% of the plant antibody or fragment thereof crosses the placenta to enter foetal circulation, relative to an equivalent mammalian produced therapeutic antibody or fragment thereof. In particular embodiments, less than 0.5%, less than 0.4%, less than 0.3%, less than 0.2% or less than 0.1% of the plant antibody or fragment thereof crosses the placenta to enter foetal circulation, relative to an equivalent mammalian therapeutic antibody or fragment thereof.

In certain embodiments, the plant antibody or fragment thereof comprises at least one plant specific glycan. As such, the glycan may be one that is not commonly found in mammals or absent in mammals. In certain embodiments, the plant specific glycan comprises a fucose with a α1-3 linkage. In certain embodiments, the plant specific glycan comprises a xylose with a β1,2 linkage. In certain embodiments, the plant specific glycan does not comprise galactose. In certain embodiments, the plant antibody comprises no fucose or xylose. In certain embodiments, the plant specific glycan comprises a fucose with a α1-3 linkage and optionally a xylose with a β1,2 linkage. In certain embodiments, the plant specific glycan comprises a fucose with a α1-3 linkage and a xylose with a β1-2 linkage. As would be appreciated by the skilled person, mammalian fucose has a α1-6 linkage.

In certain embodiments, the antibody comprises one or more tags which alter glycans, such as, for example, KDEL, OMT, ACTS, and/or VTS tags. In certain embodiments, the plant or plant cell is cotransfected or cotransformed with at least one ER chaperone, which may enhance expression. In certain embodiments, an LC glycan is removed.

The sugars and their abbreviations used herein are: Fucose (Fuc); N-acetyl Glucosamine (GlcNAc); Mannose (Man); Galactose (Gal); Hexose (Hex); β1,2-Xylose (Xyl). In certain embodiments, the plant antibody or fragment thereof has a glycopeptide profile comprising at least one glycan selected from the group consisting of: GlcNAc₂ Fuc Man₃ Xyl; GlcNAc₂ Man₃ GlcNAc; GlcNAc₂ Fuc Man₃ Xyl GlcNAc; GlcNAc₂ Man₃ Xyl GlcNAc; GlcNAc₂ Fuc Man₃ Xyl GlcNAc₂; and GlcNAc₂ Fuc Man₃ Xyl GlcNAc₂ Hex. In certain embodiments, the glycopeptide profile comprises at least one glycan of a relative percentage selected from the group consisting of: at least 1% of GlcNAc₂ Fuc Man₃ Xyl; at least 2% of GlcNAc₂ Man₃ GlcNAc; at least 2% of GlcNAc₂ Fuc Man₃ Xyl GlcNAc; at least 2% of GlcNAc₂ Man₃ Xyl GlcNAc; at least 2% of GlcNAc₂ Fuc Man₃ Xyl GlcNAc₂; and at least 0.5% GlcNAc₂ Fuc Man₃ Xyl GlcNAc₂ Hex. In certain embodiments, the glycopeptide profile comprises at least one glycan of a relative percentage selected from the group consisting of: 2-10% of GlcNAc₂ Fuc Man₃ Xyl; 2-20% of GlcNAc₂ Man₃ GlcNAc; 2-30% of GlcNAc₂ Fuc Man₃ Xyl GlcNAc; 10-40% of GlcNAc₂ Man₃ Xyl GlcNAc; 20-60% of GlcNAc₂ Fuc Man₃ Xyl GlcNAc₂; and 0.5-6% GlcNAc₂ Fuc Man₃ Xyl GlcNAc₂ Hex. In certain embodiments, the glycan is selected from the group consisting of: GlcNAc₂ Fuc Man₃ Xyl as shown in Table 4; GlcNAc₂ Man₃ GlcNAc as shown in Table 4; GlcNAc₂ Fuc Man₃ Xyl GlcNAc as shown in Table 4; GlcNAc₂ Man₃ Xyl GlcNAc as shown in Table 4; GlcNAc₂ Fuc Man₃ Xyl GlcNAc₂ as shown in Table 4; and GlcNAc₂ Fuc Man₃ Xyl GlcNAc₂ Hex as shown in Table 4.

In certain embodiments, the at least one glycan is at least 1%, 1-50%, 1-40%, 1-30%, 1-20%, 1-10%, 1-8%, 1-6%, 2-10%, 2-8% or 2-6% of GlcNAc₂ Fuc Man₃ Xyl. In certain embodiments, the at least one glycan is at least 2%, 1-60%, 1-50%, 1-40%, 1-30%, 1-20%, 2-20%, 2-18%, 2-16% or 4-15% of GlcNAc₂ Man₃ GlcNAc. In certain embodiments, the at least one glycan is at least 1%, 1-70% 1-60%, 1-50%, 1-40%, 1-30%, 2-30%, 2-26% or 2-24% of GlcNAc₂ Fuc Man₃ Xyl GlcNAc. In certain embodiments, the at least one glycan is at least 1%, 1-70% 1-60%, 1-50%,-50%, 10-50%, 1-40%, 2-40%, 5-40%, 10-40% or 15-36% of GlcNAc₂ Man₃ Xyl GlcNAc. In certain embodiments, the at least one glycan is at least 1%, 1-80%, 1-70%, 10-70%, 20-70%, 1-60%, 10-60%, 20-60%, 1-50%, 10-50%, 20-50% or 25-50% of GlcNAc₂ Fuc Man₃ Xyl GlcNAc₂. In certain embodiments, the at least one glycan is at least 0.5%, 0.5-50%, 0.5-40%, 0.5-30%, 0.5-20%, 0.5-10%, 0.5-8% or 0.5-6% of GlcNAc₂ Fuc Man₃ Xyl GlcNAc₂ Hex.

It was found that plant antibody or fragment thereof being non-oxidised may be important for binding to FcRn and potentially other Fc receptors. In certain embodiments, the plant antibody or fragment thereof is substantially non-oxidised. In certain embodiments, the substantially non-oxidised plant antibody or fragment thereof comprises at least 95% methionine and 5% or less methionine sulfoxide. In certain embodiments, the plant antibody or fragment thereof is treated with a reducing agent to provide the substantially non-oxidised antibody or fragment thereof. Reducing agents suitable for providing reduced methionine could be readily selected by the person skilled in the art. In certain embodiments, the reducing agent is selected from the group consisting of: dithiothreitol (DTI), β-mercaptoethanol (β-ME), tris(2-carboxyethyl)phosphine (TCEP), glutathione (GSH).

In a second aspect, there is provided a plant antibody or fragment thereof produced by the method of the first aspect. As such, the method may be performed as described herein and the product of the method is the plant antibody or fragment thereof.

In a third aspect, there is provided a plant produced therapeutic antibody or fragment thereof that is unable to substantially cross the placenta, comprising a glycopeptide profile comprising at least one plant glycan as described in the first aspect or elsewhere herein. As such, the at least one glycan may be, eg, at least one glycan of any of the percentages as described in the first aspect, at least one glycan as shown in Table 13, or any combination of glycans, as described in the first aspect or elsewhere herein.

In certain embodiments, the plant antibody or fragment thereof is unable to substantially bind to FcγRI, FcγRIIA, FcγRIIB, FcγRIIIA or other Fc receptors. In certain embodiments, the plant antibody or fragment thereof is able to substantially bind to FcγRn. FcγRn has been detected on the placenta and is considered essential for placenta transfer. The inability to substantially bind the above receptor(s) may be as described in the first aspect or elsewhere herein.

In certain embodiments, the plant antibody or fragment thereof is substantially non-oxidised. In certain embodiments, the substantially non-oxidised plant antibody or fragment thereof comprises at least 95% methionine and 5% or less methionine sulfoxide. In certain embodiments, the plant antibody or fragment thereof is treated with a reducing agent to provide the substantially non-oxidised antibody or fragment thereof. The reducing agent may be as described in the first aspect or elsewhere herein.

In a fourth aspect, there is provided a composition comprising the plant antibody or fragment thereof described herein and a pharmaceutically acceptable carrier. The plant antibody or fragment thereof described herein may be from, eg, the first or third aspects. The composition may be for the treatment of an autoimmune disease, infectious disease, a cancer, or a condition as described below.

In certain embodiments, the composition is suitable for oral, intradermal, intranasal, intramuscular, intraperitoneal, intravenous or subcutaneous administration. Administration can also be by continuous infusion or bolus injection. In addition, the composition can be administered in a variety of dosage forms. These include, e.g., liquid preparations and suspensions, including preparations for subcutaneous, intradermal, intramuscular, intraperitoneal, or intravenous administration (e.g., injectable administration), such as sterile isotonic aqueous solutions, suspensions, emulsions or viscous compositions that may be buffered to a selected pH. Such compositions may be formulated using a variety of pharmaceutical excipients, carriers or diluents familiar to one of skill in the art. A composition suitable for oral administration may include a variety of dosage forms, e.g., solutions, powders, suspensions, tablets, pills, capsules, caplets, sustained release formulations, or preparations which are time-released or which have a liquid filling, e.g., gelatin covered liquid, whereby the gelatin is dissolved in the stomach for delivery to the gut. Such dosage forms may include a variety of pharmaceutically acceptable excipients described herein, including but not limited to mannitol, lactose, starch, magnesium stearate, sodium saccharine, cellulose, and magnesium carbonate.

In a particular embodiment, it is contemplated herein that a composition for oral administration may be a liquid formulation. Such formulations may comprise a pharmaceutically acceptable thickening agent which can create a composition with enhanced viscosity which facilitates mucosal delivery of the active agent, e.g., by providing extended contact with the lining of the stomach. Such viscous compositions may be made by one of skill in the art employing conventional methods and employing pharmaceutical excipients and reagents, e.g., methylcellulose, xanthan gum, carboxymethyl cellulose, hydroxypropyl cellulose, and carbomer.

In a fifth aspect, there is provided the use of the plant antibody or fragment thereof described herein in the manufacture of a medicament for the treatment of cancer. The plant antibody or fragment thereof described herein may be from, eg, the first or third aspects. In certain embodiments, the medicament is suitable for oral, intradermal, intranasal, intramuscular, intraperitoneal, intravenous or subcutaneous administration, as described elsewhere herein.

In a sixth aspect, there is provided the use of the plant antibody or fragment thereof described herein in the manufacture of a medicament for the treatment of an autoimmune disease. The plant antibody or fragment thereof described herein may be from, eg, the first or third aspects. In certain embodiments, the composition is suitable for oral, intradermal, intranasal, intramuscular, intraperitoneal, intravenous or subcutaneous administration, as described elsewhere herein.

In a seventh aspect, there is provided the use of the plant antibody or fragment thereof described herein in the treatment or prevention of a condition in a pregnant mother. The plant antibody or fragment thereof described herein may be from, eg, the first or third aspects.

In an eighth aspect, there is provided a plant antibody or fragment thereof as described herein for use in treating or preventing a condition in a pregnant mother. The plant antibody or fragment thereof described herein may be from, eg, the first or third aspects.

In an ninth aspect, there is provided a method of treating a condition in a pregnant mother, comprising administering a therapeutically effective amount of the plant antibody or fragment thereof described herein to the pregnant mother. The plant antibody or fragment thereof described herein may be from, eg, the first or third aspects.

In certain embodiments of the composition, uses and methods described herein, such as in the fourth through eighth aspects, the cancer or condition is selected from the group consisting of an adrenal tumor, an acinar sarcoma, a Astrocytoma, a bladder cancer, a bone cancer, a brain spinal cord cancer, a metastatic brain tumor, a breast cancer, a carotid body tumor, a cervical cancer, a chondrosarcoma, a spinal tumor, a kidney Chronic cell carcinoma, a clear cell carcinoma, a colon cancer, a colorectal cancer, fesmoplastic small round cell tumor, a fibroproliferative small round cell tumor, an extra-bone Myxoid chondrosarcoma, a fibrosarcoma, a fibrous dysplasia, a gallbladder or cholangiocarcinoma, a pregnancy trophoblastic disease, a germ cell tumor, a neck cancer, a hepatocellular carcinoma, an islet cell Tumor, Kaposi's sarcoma, a kidney cancer, a leukemia, a liposarcoma/malignant fat Adenoma, a liver cancer, a lymphoma, a lung cancer, a stromal cell tumor, a melanoma, a meningioma, a multiple endocrine tumor, a multiple myeloma, a myelodysplastic syndrome, a nerve A blastoma, a neuroendocrine tumor, an ovarian cancer, a pancreatic cancer, a papillary thyroid cancer, a parathyroid tumor, a peripheral schwannomas, a pituitary tumor, a prostate cancer, a posterior uveal melanoma, a primary central nervous system tumora renal metastasis cancer, a rhabdoid tumor, a rhabdomyosarcoma, a sarcoma, a skin cancer, a soft tissue sarcoma, a squamous cell carcinoma, a stomach cancer, A synovial sarcoma, a testicular cancer, a thymic carcinoma, a thymoma, a metastatic thyroid cancer, and a uterine cancer.

In certain embodiments of the composition, uses and methods described herein, such as in the fourth through eighth aspects, the autoimmune disease or condition is selected from the group consisting of: type I diabetes mellitus (T1D), Crohn's disease, ulcerative colitis, myasthenia gravis, vitiligo, Graves' disease, Hashimoto's disease, Addison's disease, autoimmune gastritis, autoimmune hepatitis, rheumatoid disease, systemic lupus erythematosus, progressive systemic sclerosis and variants, polymyositis, dermatomyositis, primary biliary cirrhosis, autoimmune thrombocytopenia, Sjogren's syndrome, multiple sclerosis and psoriasis.

As used herein, the term “autoimmune disease” means a disease resulting from an immune response against a self tissue or tissue component, including both self antibody responses and cell-mediated responses. The term autoimmune disease, as used herein, encompasses organ-specific autoimmune diseases, in which an autoimmune response is directed against a single tissue, such as type I diabetes mellitus (T1D), Crohn's disease, ulcerative colitis, myasthenia gravis, vitiligo, Graves' disease, Hashimoto's disease, Addison's disease and autoimmune gastritis and autoimmune hepatitis. The term autoimmune disease also encompasses non-organ specific autoimmune diseases, in which an autoimmune response is directed against a component present in several or many organs throughout the body. Such autoimmune diseases include, for example, rheumatoid disease, systemic lupus erythematosus, progressive systemic sclerosis and variants, polymyositis and dermatomyositis. Additional autoimmune diseases include pernicious anemia including some of autoimmune gastritis, primary biliary cirrhosis, autoimmune thrombocytopenia, Sjogren's syndrome, multiple sclerosis and psoriasis. The person skilled in the art would understand that the composition, uses and methods described herein can be applied to these or other autoimmune diseases, as desired.

In certain embodiments of the above aspects, the pregnant mother is an animal. For example, in particular embodiments, the pregnant mother is dog, cat, horse, cow or bird.

### C. Methods of Producing Plant Antibodies

The plant antibody or fragment thereof of the present disclosure may be made using recombinant techniques as described elsewhere in this disclosure, eg, in the first aspect, and as further described below.

In certain embodiments, at least one polynucleotide molecule encoding a polypeptide sequence of the antibody or fragment thereof could be cloned into an expression vector that would be transcribed when transfected or transformed into a plant or plant cell. In embodiments, an expression vector may comprise a plasmid, plant virus, or plant retrovirus amongst others. The at least one polynucleotide molecule can be isolated using standard molecular biology approaches, for example by using polymerase chain reactions to produce the at least one polynucleotide molecule, which is then purified and cloned into an expression vector and transfected or transformed into a plant or plant cell. Additional techniques useful in the practice of this disclosure may be found in Current Protocols in Molecular Biology 2007 by John Wiley and Sons, Inc.; Molecular Cloning: A Laboratory Manual (Third Edition) Joseph Sambrook, Peter MacCallum Cancer Institute, Melbourne, Australia; David Russell, University of Texas Southwestern Medical Center, Dallas, Cold Spring Harbor.

Selecting a particular at least one polynucleotide molecule of the present disclosure to encode an antibody or fragment thereof of the disclosure is well within the skill in the art. For example, codon usage tables for a particular species may be used to generate a reverse complement that encodes a polypeptide of the present disclosure. See, for example, The Sequence Manipulation Suite: JavaScript programs for analyzing and formatting protein and DNA sequences. Biotechniques 28:1102-1104 (bioinformatics.org/sms2/rev_trans.html) or the Codon Usage Table: From the codon usage database (http://www.kazusa.or.jp/codon/): Homo sapiens [gbpri]: 93487 CDS's (40662582 codons). Alternatively, codon frequencies can be optimized for use in plants using frequency data such as that available from various codon usage records. One such record is the Codon Usage Database. Y. Nakamura et al., “Codon usage tabulated from the international DNA sequence databases: status for the year 2000.” Nucl. Acids Res. 28, 292 (2000). Using these techniques, one skilled in the art readily can generate a “codon optimized” polynucleotide sequence that encodes a polypeptide sequence of the antibody or fragment thereof of the present disclosure.

In further embodiments, the polynucleotides can be operably joined to a promoter. Expression in prokaryotic hosts can be accomplished using prokaryotic regulatory regions. Expression in eukaryotic hosts can be accomplished using eukaryotic regulatory regions. Such regions will, in general, include a promoter region sufficient to direct the initiation of RNA synthesis. In embodiments, the polynucleotide molecule can further comprise transcriptional and translational regulatory sequences, depending upon the nature of the host. The transcriptional and translational regulatory signals may be obtained or derived from viral sources, such as a retrovirus, adenovirus, bovine papilloma virus, simian virus, or the like.

In embodiments, at least one polynucleotide is inserted into a vector capable of integrating the desired sequences into the host cell genome, such as the nuclear genome or mitochondrial genome. Additional elements may also be needed for optimal synthesis of the mRNA. These elements may include splice signals, as well as transcription promoters, enhancers, and termination signals and are all within the art.

Preferred plant vectors are pTRA-k vectors. Other suitable vectors will be readily apparent to the skilled artisan, for example, potato virus X (PVX) expression vectors and PVX-based vectors, Tobacco mosaic virus (TMV) expression vectors and TMV-based vectors, (see, eg, Dickmeis et al. Biotechnology Journal 9:1369-1379 (2014). Preferably, at least one polynucleotide is incorporated into a plasmid or viral vector capable of autonomous replication in the recipient host. Any of a wide variety of vectors may be employed for this purpose, such as prokaryotic and eukaryotic vectors. The eukaryotic vectors can be viral vectors. For example, and not by way of limitation, the vector can be a potato virus X or tobacco mosaic virus or any of a number of other vectors. The viral vectors include either DNA or RNA viruses to cause expression of the insert DNA or insert RNA. Alternatively, methods of introduction into the host cell can be performed by a variety of well-known methods, i.e., transformation, transfection, conjugation, protoplast fusion, electroporation, calcium phosphate-precipitation, direct microinjection, and the like. These techniques are all within the art. See, for example, Current Protocols in Molecular Biology 2007 by John Wiley and Sons, Inc.; Molecular Cloning: A Laboratory Manual (Third Edition) Joseph Sambrook, Peter MacCallum Cancer Institute, Melbourne, Australia; David Russell, University of Texas Southwestern Medical Center, Dallas, Cold Spring Harbor. Additionally, polynucleotides of the antibody or fragment thereof of the disclosure can be directly injected into cells or may be impelled through cell membranes after being adhered to microparticles or nanoparticles, such as the synthetic nanocarriers.

In certain embodiments, the at least one polynucleotide sequence encodes a polypeptide sequence having at least 70%, at least 75%, at least 80%, at least 85%, at least 90%, at least 95%, at least 96%, at least 97%, at least 98%, at least 99% identity or 100% identity to a sequence selected from one or more of known antibody sequences corresponding to any one of the antibodies described in Table 1 or Table 2. Unless otherwise described, variants (such as those above having less than 100% sequence identity) of the at least one polynucleotide sequence or polypeptide sequence retain the ability of the wild type protein from which the variant was derived, although the activity may not be at the same level. In preferred embodiments, the variants have at least about 50%, at least about 60%, at least about 70%, at least about 80%, at least about 90%, at least about 100% efficacy compared to the original sequence. In preferred embodiments, the variant has improved activity as compared to the original sequence. For example, variants with improved activity have at least about 105%, at least about 110%, at least about 120%, at least about 130%, at least about 140%, at least about 150%, or at least about 160% efficacy compared to the original sequence. Variants can be identified by the person skilled in the art by conducting BLAST searches using the disclosed sequences, or literature searches using gene, enzyme, substrate or product names, and those variants can be tested using the methods of the Examples disclosed herein.

Recombinant production of the antibody or fragment thereof of the present disclosure may be produced in several ways using cells plants. Appropriate culture mediums and conditions for the above-described host cells are known in the art. In certain embodiments, the method of recombinant production involves co-infiltration of the p19 suppressor of gene silencing from, eg, Tomato bushy stunt virus, potato virus X or cucumber necrosis virus, with at least one polynucleotide encoding the antibody or fragment thereof. In certain embodiments, plant optimized vectors may be used comprising the at least one polynucleotide encoding the antibody or fragment thereof. These techniques are all within the art. See, for example, Rosenberg et al. PLoS One 8:e58724 (2013) or Diamos et al. Front. Bioeng. Biotechnol. 7(472)1-15 (2020).

Production of the plant antibody or fragment thereof using the methods described herein provides high levels of the antibody or fragment thereof. The plant antibody or fragment thereof may be separated from crude protein extracts by methods known in the art, such as by affinity chromatography. Alternatively, the antibody or fragment thereof can be recovered and purified from recombinant cell cultures by well-known methods including ammonium sulfate or ethanol precipitation, Protein A, acid extraction, anion or cation exchange chromatography, phosphocellulose chromatography, hydrophobic interaction chromatography, affinity chromatography, hydroxylapatite chromatography, and lectin chromatography or combinations thereof. In particular embodiments, high performance liquid chromatography (“HPLC”) is employed for purification. For example, Protein A and a second chromatography (eg MEP) column. The person skilled in the art would readily understand how to purify a plant antibody and could also refer to publications, such as Zarrineh, M et al. Analytical Biochemistry 113909 (2020); or Rosenberg et al. PLoS One 8:e58724 (2013) or Diamos et al. Front. Bioeng. Biotechnol. 7(472)1-15 (2020).

In some embodiments, expression level of the antibody or fragment thereof produced in N. tabacum and N. benthamiana, are in the range 100-2000 mg/kg per leaf biomass, such as 200-2000 mg/kg per leaf biomass, eg, 400-1400 mg/kg per leaf biomass, or at least 500 mg/kg per leaf biomass, In some embodiments, processed leaf extracts contain the antibody or fragment at 0.2% w/w to 2% w/w. In other embodiments, affinity chromatography is conducted using passage over a column to purify the antibody or fragment to >95% purity. In other preferred embodiments, purified the antibody or fragment is obtained at >75% purity, >80% purity, >85% purity, >90% purity, >95% purity, >96% purity, >97% purity, >98% purity, >99% purity, and/or at 100% purity.

In one preferred embodiment, chitosan can be used in the processing of the leaf extract to clarify plant extracts. In a further preferred embodiment, collagen is added to the leaf extract and purified the antibody or fragment to even further increase thermal stability and shelf life at extreme temperatures.

In some embodiments the plant antibody or fragment is maintained in a liquid, frozen or powdered form.

Moreover, the disclosure additionally encompasses a plant antibody or fragment which is differentially modified during or after translation, e.g., by acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, specific chemical cleavage by cyanogen bromide, trypsin, chymotrypsin, papain, V8 protease, NaBH.sub.4, acetylation, formylation, oxidation, reduction, metabolic synthesis in the presence of tunicamycin, etc.

Additional post-translational modifications encompassed by the present disclosure include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression.

Nicotiana plant species, including tobacco, have been identified as a cost-effective means for the expression of large quantities of the antibody or fragment.

### EXAMPLES

### Example 1. Placental Transfer

An experiment was conducted to analyse transfer of human mAbs produced in mammalian and plant cells across the placenta of rhesus macaques (Macaca mulatta) in the days prior to parturition. mAbs produced in mammalian (CHO, HEK 293) or plant (Nicotiana benthamiana) cells were injected passively by the subcutaneous (SC) route to a total of six M. mulatta dams close to parturition, as determined by sonigram, all receiving 5 mg/kg of each Ab. The antibodies included plant produced Nb PGT121, Nb PGT121-YTE and Nb VRC07-523-LS and the mammalian antibodies Mam PGT121 and Mam 830A IgG1. The length of time that the mAb was in the dam was variable, due to not knowing the precise time of parturition, and wanting to allow birth to proceed naturally (vaginally). Antibody concentrations in the dam and in their infants were determined at multiple time points by IL-2 ELISA using standard curves for the matched antibodies. This variation in time since SC infusion is one reason for variability in the concentrations in different animals, ranging from 3.8 to 24.77. The data shown below summarize the comparison for matched time points in dam and infant. In total, six pregnant dams were injected with six plant mAbs and three control mammalian mAbs.

A summary of the antibodies used and the day of administration is shown in Table 3 (below). In experiments 1 through 4, a single antibody was administered to dams prior to birth. Experiments 5 and 6 co-administered 2 and 3 antibodies, respectively. The day of infusion, such as “−6”, means infusion 6 days before birth.

### Results

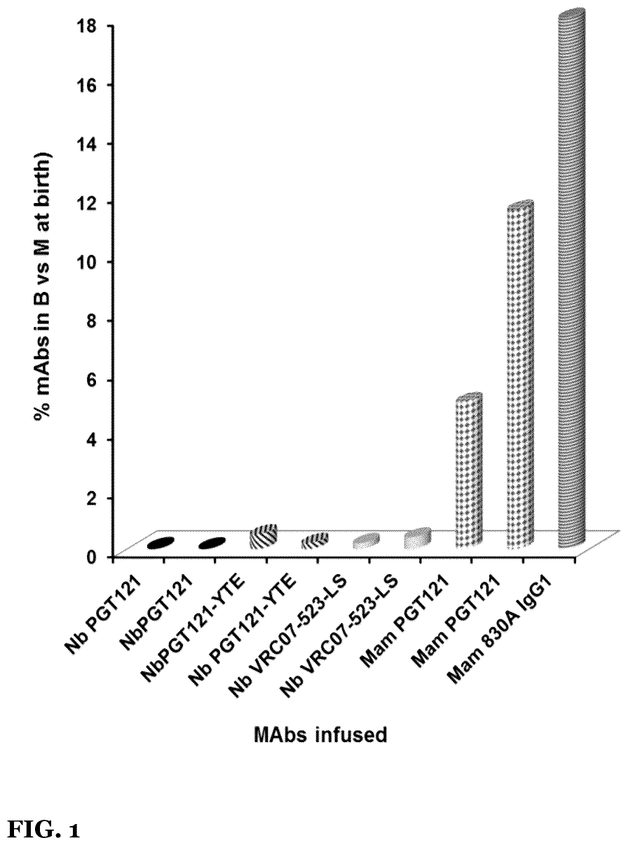

A summary of the results is shown in Table 3 (below) and are illustrated in FIG. 1.

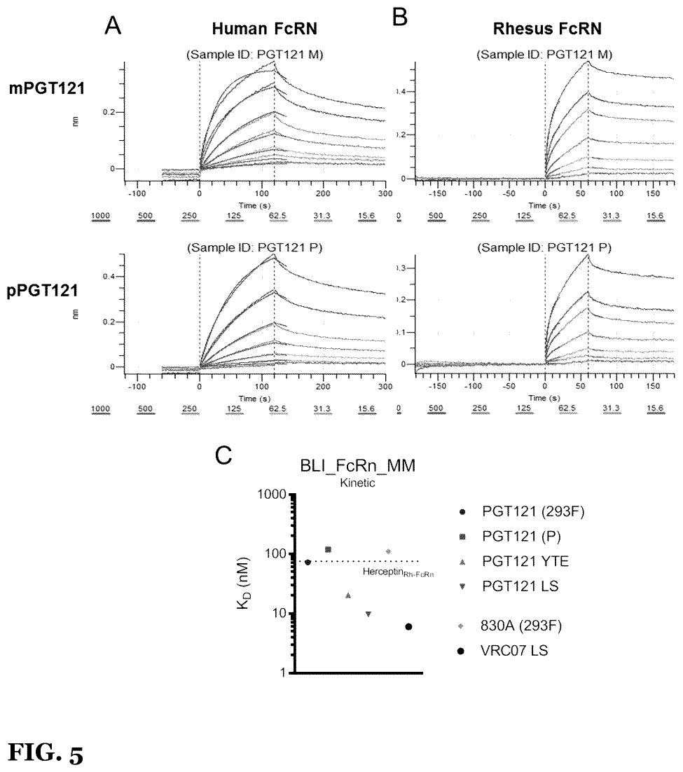

FIG. 1 shows percent mAb levels in six mother-baby pairs at birth. Mothers were infused with one, two or three mAb (see Table 1) and the infused antibodies indicated on the X-axis were compared in the babies and paired mothers at birth and presented as a percentage on the Y-axis. While the YTE and LS mutants with increased affinity for FcRn (see FIG. 5) very slightly improved transfer, only mammalian mAbs were significantly transferred across the placenta. Nb: Nicotiana benthamiana. Mammalian (Mam) mAbs include PGT121 produced in CHO cells and 830A produced in HEK293 cells.

TABLE 3
SUMMARY OF DAM/BABY PLACENTAL TRANSFER EXPERIMENTS

| | | | | | Dam mAb | Baby mAb | |
|---|---|---|---|---|---|---|---|
| | | | | | levels | levels at | |
| | | | | Day(s) of | at birth | birth | |
| Exp | Dam# | Baby# | mAb infusion | infusion | (ug/ml) | (ug/ml) | FIG. |
| 1 | 001 | 001.1 | Nb PGT121-YTE | −15, −1 | 19.6 | 0.1 | 1, 2 |
| 2 | 002 | 002.1 | Nb PGT121-YTE | −9, −5 | 28.5 | 0.001 | 1, 2 |
| 3 | 003 | 003.1 | Nb PGT121 | −4 | 5.25 | 0.003 | 1, 2 |
| 4 | 004 | 004.1 | Nb PGT121 | −5, −1 | 18.53 | 0.011 | 1, 2 |
| 5 | 005 | 005.1 | Mam PGT121 | −6 | 13.00 | 0.71 | 1 |
| | | | Nb VRC07-523-LS | −6 | 3.79 | 0.01 | |
| 6 | 006 | 006.1 | Nb VRC07-523-LS | −6 | 3.95 | 0.02 | 3 |
| | | | Mam PGT121 | −6 | 12.77 | 1.47 | |
| | | | Mam 830A | −6 | 19.17 | 3.5 | |

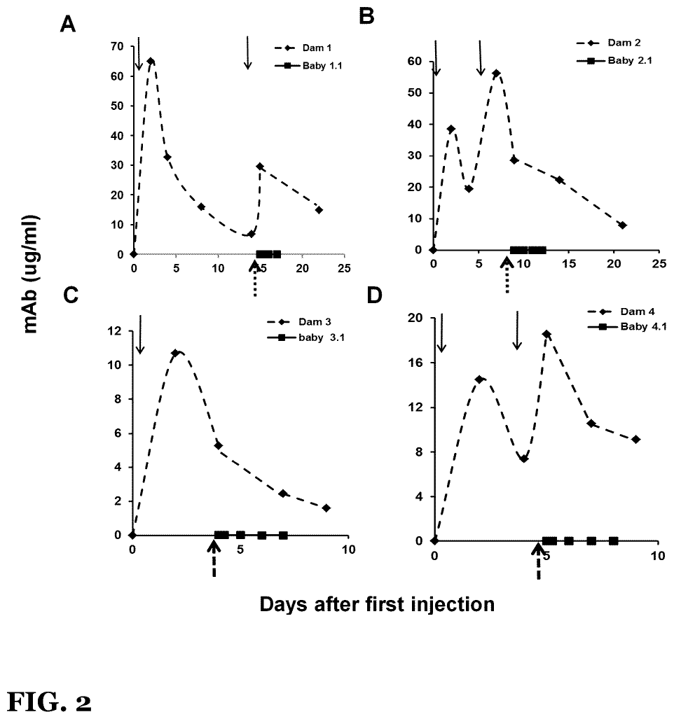

The results of experiments 1-4 are shown in FIG. 2A-D and in Table 4, Table 5, Table 6, and Table 7. Pharmacokinetics of infused with 5 mg/kg of plant PGT121 in four mothers (blue diamonds) and their babies (red squares). 1 and 2: mothers received PGT-YTE and 3 and 4 received WT PGTG121. Downward arrows shown times of PGT121 injection and upward arrows indicate day of birth. Some mothers received two injections to maintain high levels of the infused antibody.

Table 4 and FIG. 2A show the results of experiment #1 from Table 3, in which plant produced Nb PGT121-YTE was administered at −15 and −1 days before birth. The results show significant levels of antibody in the dam and undetectable levels in the baby (ie 0).

TABLE 4
Experiment #1 Nb PGT121-YTE
Antibody Levels in DAM and Baby

| Day | Dam | Baby |
|---|---|---|
| 0 | 0 | |
| 2 | 64.9 | |
| 4 | 32.7 | |
| 8 | 15.9 | |
| 14 | 6.8 | |
| 15 | 29.5 | 0 |
| 15.25 | | 0 |
| 16 | | 0 |
| 17 | | 0 |
| 18 | | |
| 22 | 14.89 | |
| 29 | 5.8 | |

Table 5 and FIG. 2B show the results of experiment #2 from Table 3, in which plant produced Nb PGT121-YTE was administered at −9 days before birth. The results show significant levels of antibody in the dam and undetectable levels in the baby (ie 0).

TABLE 5
Experiment #2 Nb PGT121-YTE
Antibody Levels in Dam and Baby

| Day | Dam | Baby |
|---|---|---|
| 0 | 0 | |
| 2 | 38.5 | |
| 4 | 19.5 | |
| 7 | 56.3 | |
| 9 | 28.5 | 0 |
| 9.25 | | 0 |
| 10 | | 0 |
| 11 | | 0 |
| 12 | | 0 |
| 14 | 22.2 | |
| 21 | 7.78 | |

Table 6 and FIG. 2C show the results of experiment #3 from Table 3, in which plant produced Nb PGT121 was administered at −4 days before birth. The results show significant levels of antibody in the dam and very low levels in the baby (eg Momo-0.003).

TABLE 6
EXPERIMENT #3 NB PGT121-YTE
ANTIBODY LEVELS IN DAM AND BABY

| Day | Dam | Baby |
|---|---|---|
| 0 | 0 | |
| 2 | 10.7 | |
| 4 | 5.28 | 0.003 |
| 4.25 | | 0.003 |
| 5 | | 0.003 |
| 6 | | 0.002 |
| 7 | 2.43 | 0.002 |
| 9 | 1.59 | |

Table 7 and FIG. 2D show the results of experiment #4 from Table 3, in which plant produced Nb PGT121 was administered at −4 days before birth. The results show significant levels of antibody in the dam and very low levels in the baby (eg Momo-0.003).

TABLE 7
EXPERIMENT #4 NB PGT121-YTE
ANTIBODY LEVELS IN DAM AND BABY

| Day | Dam | Baby |
|---|---|---|
| 0 | 0 | |
| 2 | 10.7 | |
| 4 | 5.28 | 0.003 |
| 4.25 | | 0.003 |
| 5 | | 0.003 |
| 6 | | 0.002 |
| 7 | 2.43 | 0.002 |
| 9 | 1.59 | |

Table 8 shows the results of experiment #5 from Table 3. Specifically, placental transfer of mAb to babies from a macaque injected with 5 mg/kg of the two different mAbs 6 days prior to birth. Mother #5 and baby #5.1 were assayed for the presence of Nb VRC07-523-LS, and mammalian PGT121. Nb: N. benthamiana, mammalian: HEK293. The results show significant levels of mammalian PGT121 and Nb VRC07-LS antibody in the dam, low levels mammalian PGT121 in the baby (eg 0.45-0.71) and very low levels of Nb VRC07-LS in the baby (eg 0.0.1).

TABLE 8
EXPERIMENT #5 MAMMALIAN PGT121 AND
NB VRC07-LS ANTIBODY LEVELS IN DAM AND BABY

| Day | Dam 121 m | Baby 121 m | Dam VRC | Baby VRC |
|---|---|---|---|---|
| 0 | 0 | | 0 | |
| 2 | 21.9 | | 7.14 | |
| 5 | 18.81 | | 5.4 | |
| 6 | 13.46 | 0.71 | 3.79 | 0.01 |
| 6.25 | | 0.6 | | 0.01 |
| 7 | 12.07 | 0.55 | 3.37 | 0.01 |
| 8 | | 0.51 | | 0.01 |
| 9 | 12.28 | 0.45 | 3.33 | 0.01 |
| 14 | 8.2 | | 2.39 | |

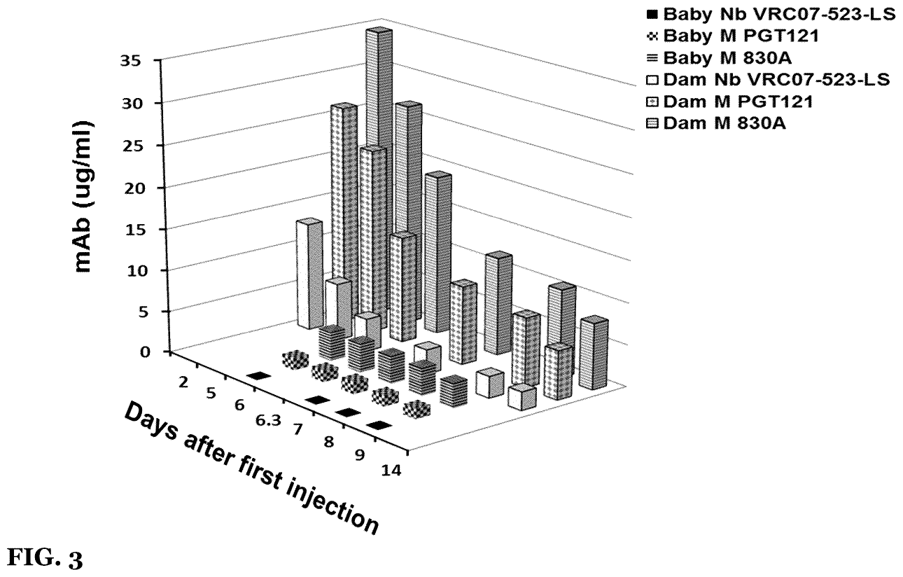

Table 9 and FIG. 3 show the results of experiment #6 from Table 3. Specifically, placental transfer of mAb to babies from a macaque injected with 5 mg/kg of each of the three different mAbs 6 days prior to birth. Macaque #6 and baby #6.1 were assayed for the presence of Nb VRC07-523-LS, and mammalian PGT121 and 830A. Nb: N. benthamiana, mammalian: HEK293. The results show significant levels of plant produced Nb VRC07-LS antibody in the dam and very low levels in the baby (eg 0.02), compared to higher levels of mammalian PGT121 and 830A in the dams and higher levels in the babies (eg 1.22-1.47 and 3-3.5, respectively). As such, the mammalian antibodies crossed significantly and little if any of the plant antibodies crossed into the baby.

TABLE 9
EXPERIMENT #6 NB VRC07-LS, MAM PGT121
& MAM 830A ANTIBODY LEVELS IN DAM AND BABY

| | | | Baby | Dam | | Dam |
|---|---|---|---|---|---|---|
| Day | Baby VRC | Baby 121 | 830A | VRC | Dam 121 | 830A |
| 2 | | | | 13.01 | 26.21 | 34.53 |
| 5 | | | | 6.91 | 22.14 | 26.61 |
| 6 | 0.02 | 1.47 | 3.5 | 3.95 | 12.77 | 19.17 |
| 6.3 | | 1.37 | 3.45 | | | |
| 7 | 0.02 | 1.45 | 3.37 | 2.93 | 9.43 | 11.79 |
| 8 | 0.02 | 1.3 | 3.36 | | | |
| 9 | 0.02 | 1.22 | 3 | 2.59 | 8.39 | 10.61 |
| 14 | | | | 2.2 | 5.85 | 7.96 |

### Example 2. Comparison of Plant and Mammalian PGT121 Binding to Macaque FcRgRIIa, FcRgIIb and FcgRIII Using Biolayer Interferometry (BLI

As the plant antibodies in Example 1 were found not to cross the placenta, binding to Fc receptors was examined.

Affinity (BLI): Using the method of Boesch et al. MABS 9(3):455-465 (2017) His-tagged Rhesus macaque Fc gamma receptors (I, Ha, IIb, and IIIa) were prepared in assay wash buffer (PBS, 0.05% Tween20, 0.1% BSA) at 100 nM concentration. Antibodies were diluted in assay wash buffer for a 2× titration series starting at 62.5 nM for rhesus FcgR1 and 1000 nM for rhesus FcgRIIa, FcgRIIIb, and FcgRIIIa. High precision SAX2 biosensors (Fortebio #18-5136) were conjugated with biotin-anti-penta-His antibody (Thermo #MA1-135). The affinity experiment was performed such that the anti-his loaded biosensors were saturated with his-tagged Fc gamma receptors and then subjected to wells containing a titration of the antibody under investigation. Receptor—antibody kinetics were measured, the biosensor was regenerated to remove the Fc gamma receptors, and the process was repeated. Association and dissociation curves were plotted.

Phagocytosis Assay: According to Chan et al. (J Immunol. 197(7):2936-47, 2016) fluorescent polystyrene beads (Life technologies #F8823) were conjugated with Bal gp120 protein via crosslinking carboxylic bead surface with primary amines on Bal gp120. Assay plates were prepared with antibodies titrations at 5× dilutions from 25,000 ng/ml to 0.32 ng/ml, in THP-1 media (RPMI+10% FBS). Bal gp120 coupled beads and THP-1 cells were added to the assay plate at a 10:1 bead to cells ratio for a total of 20,000 cells. The assay plate was incubated at 37° C. under 5% CO₂ for 4 hours. Cells were washed in cold PBS, fixed with 4% paraformaldehyde, washed, and analysed on a flow cytometer.

Flow cytometry analysis included gating on cell populations in the FSC vs SSC plot, followed by exporting the FITC+signal's median fluorescent intensities (MFI). A phagocytosis score was calculated by multiplying the MFI value with the percent FITC positive and plotted.

### Results

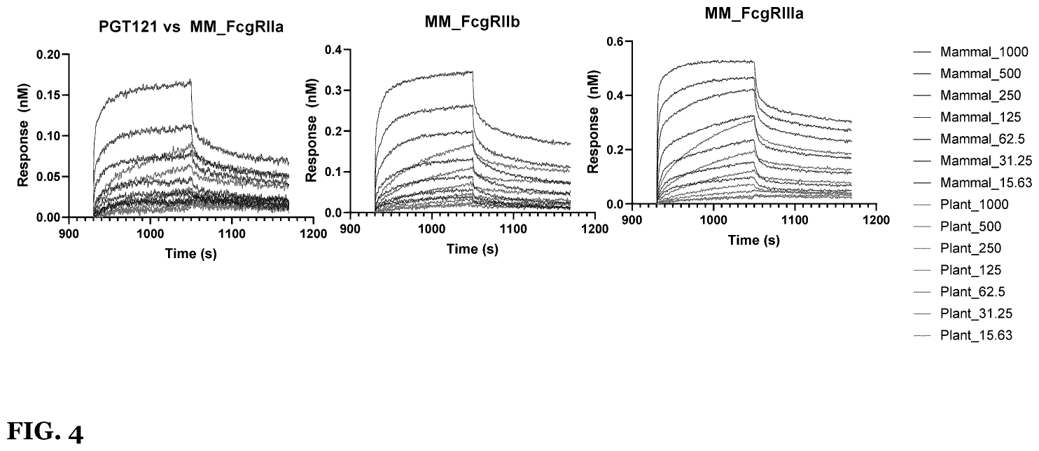

FIG. 4 shows a comparison of plant and mammalian PGT121 binding to macaque FcRgRIIa, FcRgIIb and FcgRIII using biolayer interferometry (BLI), receptor on solid phase as described by Chan et al, 2016.

FIG. 5 shows a comparison of SPR response curves and equilibrium binding constants (K_{D}) of plant and mammalian PGT121 to human and macaque FcRn. mAbs were directly printed onto the SPR chip and analyzed for binding to human (A) and rhesus macaque (B) over the range of 1,000-15.6 nM. (C) Comparison of the equilibrium binding constants (K_{D}) measured by SPR of plant-derived PGT121 YTE and LS Fc mutants to macaque FcRn confirming the higher binding of the engineered YTE and LS mutants. Wild type antibodies included plant-derived PGT121 and VRC07-523-LS, and mammalian (CHO)-derived PGT121 and HEK293-derived 830A and Herceptin. Plant PGT121 bound to both human and macaque FcRn, with better binding to the macaque FcRn than the mammalian PGT121.

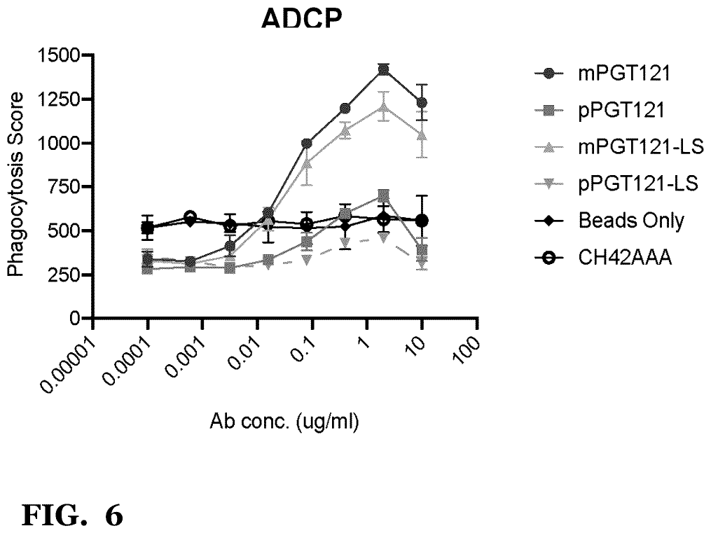

FIG. 6 shows a comparison of the ability of plant and mammalian PGT121 to induce FcR effector function as measured using an antibody-mediated phagocytosis (ADCP) assay. (Chan et al, 2016). Plant PGt121 was less efficient in this functional phagocytosis assay than Man PGT121 reflecting reduced binding to FcRs.

The data show that plant antibodies bind human and macaque FcRn. FcRN is responsible for retention of antibodies in foetal circulation. YTE and LS mutant antibodies were made to increase binding to FcRN and to help the antibodies cross the placenta into foetal circulation. These mutations did increase affinity for FcRN. The results demonstrated that despite binding to FcRN, the plant antibodies did not cross the placenta to enter foetal circulation.

### Example 3. FcγR Engagement mAb Panel

Antibody binding to human Fc receptors was measured using an IL-2 ELISA. This assay differs from the above binding assay (BLI assay) of Example 2, because this assay involves cell triggering and IL-2 production. ie cell activation. The assay uses Fc receptor with CD₃ signalling sequence—therefore indicating whether the Fc is triggered. The antibodies tested were plant produced Nb PGT121-LS, Nb PGT121-YTE, Nb PGT121, Nb PGT121-KDEL and Nb VRC01-KDEL, and mammalian PGT121, VRC07-523, VRC07-523-LS, 830A, with a LS HIG positive control.

IL-2 production assays: As an alternative method for assessing FcR binding, we utilized a method previously described by Corrales-Aguilar, et al. utilizing a panel of BW5147 (mouse T cell lymphoma) cells which have been stably transfected to express a chimeric receptor containing a rhesus macaque FcγR (FcRI, FcRIIA, FcRIIB, or FcRIII) ectodomain and the CD₃ζ intracellular signaling domain (received from Hartmut Hengel, Philipp Kolb, and team). This chimeric FcR leads to IL-2 secretion upon engagement with IgG with the corresponding Fc characteristics. Parental BW5147 cells without FcR-CD₃ expression were used as a negative control to assess background IL-2 secretion.

In brief, high-binding 96-well plates (vendor info) were coated with a 3-fold serial dilution starting at 50 μg/mL of each monoclonal antibody of interest in 0.1 M sodium bicarbonate buffer overnight at 4° C. Then, 50,000 BW5147 FcR-CD₃ζ cells were added and cultured in RPMI (vendor) supplemented with 10% (v/v) FBS (vendor), sodium pyruvate (vendor), and β-mercaptoethanol (Gibco) for 24 h at 37° C. and 5% CO₂. At the time that cells were added, 384-well high binding plates (Corning 3700) was coated with 3 μg/mL unconjugated rat anti-mouse IL-2 (vendor) overnight at 4° C. Plates were washed 1× with 1×PBS (vendor) with 0.1% Tween-20 (vendor) using an automatic plate washer (BioTek) and blocked for 1-2 h with [Superblock info] at RT. Following another 1× wash, supernatant from the BW5147 FcR-CD₃ cultures was added directly to the IL-2 detection ELISA in duplicate and incubated at RT for 1-2 h. Mouse IL-2 diluted in BW5147 culture medium was used as a positive control. Plates were washed 1×, and biotin rat anti-mouse IL-2 secondary (vendor) was added and incubated for 1 h at RT. Following another 1× wash, streptavidin-HRP tertiary (vendor) was added and incubated for 30 min at RT. Plates were washed 2×, and SureBlue [info] was added for 3-5 min in the dark at RT. An equal volume of TMB Stop Solution was then added, plates were shaken for 5 sec, and optical density at 450 nm was read using a SpectraMax plate reader. OD450 from the ELISA to detect IL-2 in the cell supernatant is shown on the y-axis and log of the concentrations of the antibodies on the x-axis of the plots.

### Results

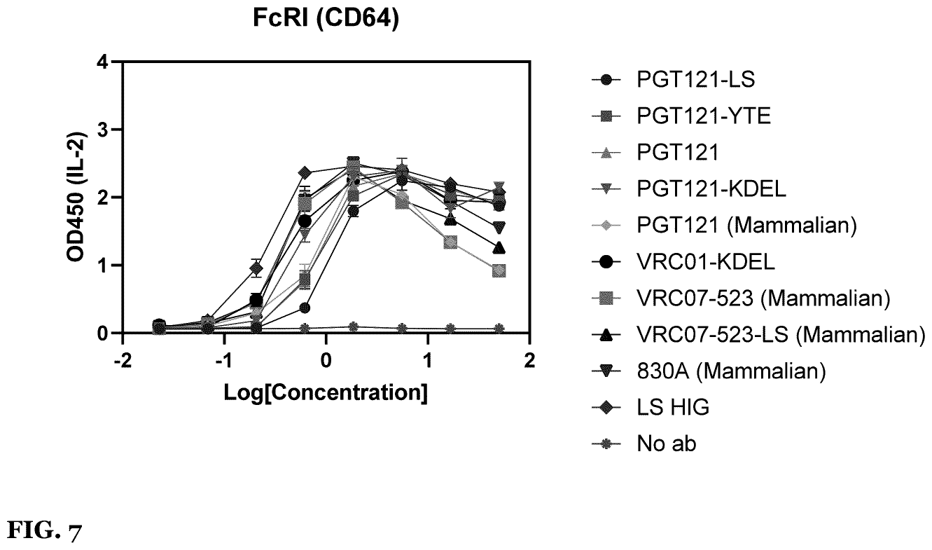

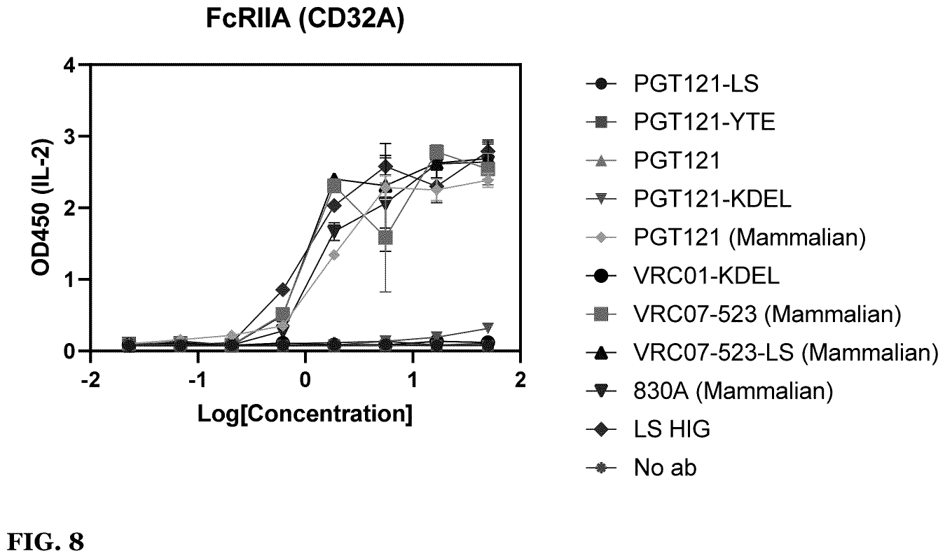

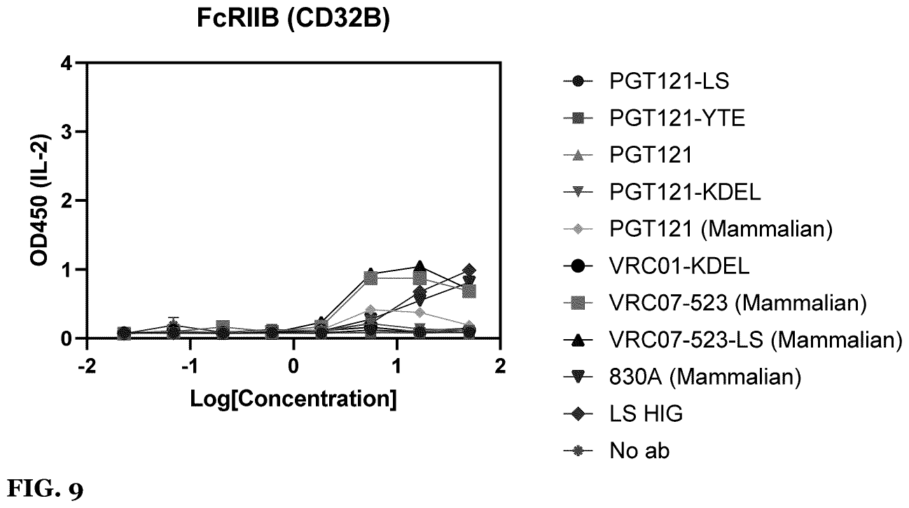

The results are shown in FIGS. 7-11 and Tables 10-13. FIG. 7 and Table 10 show binding to FcγRI (CD64) and the results confirm that plant mAbs bind and trigger IL-2 production in BW cells transfected with FcγRI, although at a slower rates than the mammalian counterparts. FIG. 8 and Table 11 show binding to FcγRIIA (CD₃₂A) and the results show that mammalian and control antibodies but not plant antibodies bound FcγRIIA and trigger IL-2 production in BW cells transfected with FcγRIIA. FIG. 9 and Table 12 show binding to FcγRIIB (CD₃₂B) and the results show that only mammalian antibodies bound FcγRIIB.

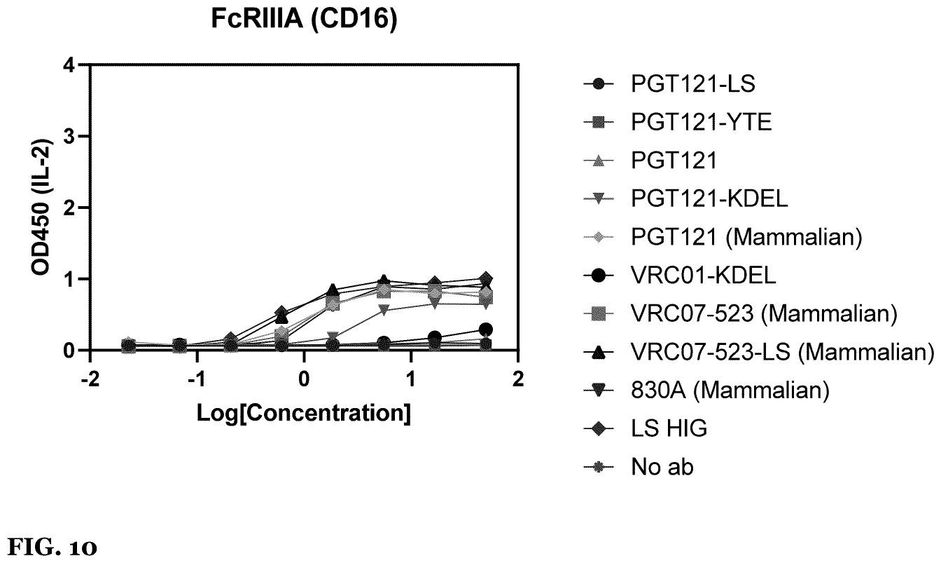

FIG. 10 and Table 13 show binding to FcγRIII (CD16) and the results show that all WT plant produced antibodies did not bind FcγRIIIA (CD16), whilst the mammalian antibodies, positive control and Nb PGT121-KDEL did bind. Addition of the ER-retrieval KDEL tag, resulted in a Nb PGT121-KDEL 4Ab lacking the complex glycans fucose or xylose and recovery of significant binding to FcγRIIIA and IL-2 production.

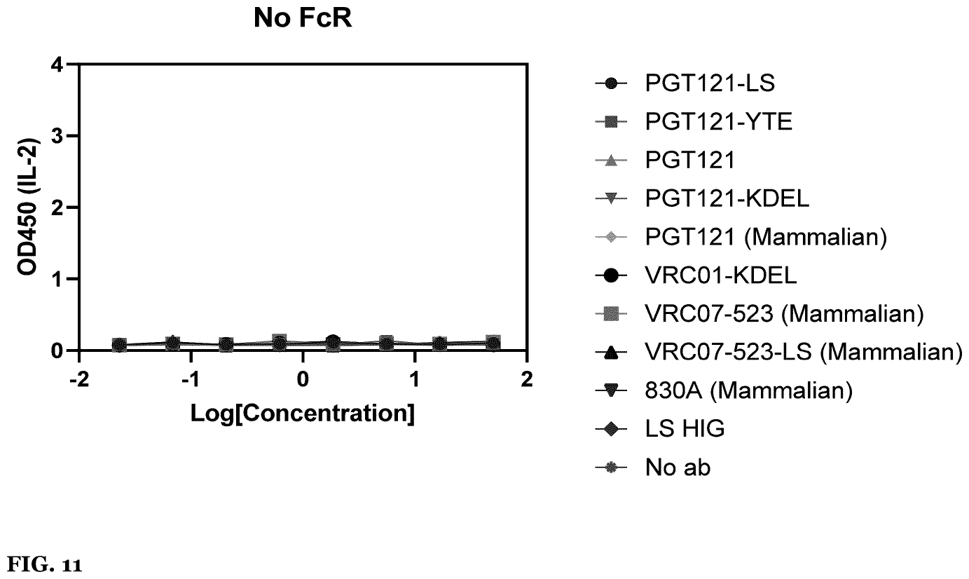

FIG. 11 shows the no FcγR negative control and no antibodies bound. The results show that all antibodies bound FcγRI (CD64). All plant produced antibodies did not bind FcγRIIA (CD₃₂A) and FcγRIIB (CD₃₂B), whilst the mammalian antibodies and positive control did bind. All plant produced antibodies except Nb PGT121-KDEL did not bind FcγRIII (Ca), whilst the mammalian antibodies, positive control and Nb PGT121-KDEL did bind. Accordingly, despite the mutations to enhance plasma transfer, the plant antibodies still did not bind FcγRIIA (CD₃₂A) and FcγRIIB (CD₃₂B).

TABLE 10
FcyRI(CD64)
EC50 >50 denotes samples where EC50 could not
be calculated due to flat curve.

| Antibody | EC50 (ug/mL) | AUC |
|---|---|---|
| Nb PGT121-LS | 1.200 | 3.656 |
| Nb PGT121-YTE | 0.7347 | 3.989 |
| Nb PGT121 | ~0.7100 | 4.072 |
| Nb PGT121-KDEL | ~0.5481 | 4.463 |
| PGT121 (Mammalian) | 0.6186 | 3.550 |
| Nb VRC01-KDEL | 0.4190 | 4.689 |
| VRC07-523 (Mammalian) | 0.3262 | 4.145 |
| VRC07-523-LS(Mammalian) | 0.3035 | 4.371 |
| 830A (Mammalian) | 0.3981 | 4.848 |
| LS HIG | 0.2329 | 5.555 |
| No ab | >50 | 0.2336 |

TABLE 11
FcγRIIA (CD32A)
EC50 >50 denotes samples where EC50 could not
be calculated due to flat curve.

| Antibody | EC50 (ug/mL) | AUC |
|---|---|---|
| Nb PGT121-LS | >50 | 0.2613 |
| Nb PGT121-YTE | >50 | 0.2465 |
| Nb PGT121 | >50 | 0.2510 |
| Nb PGT121-KDEL | >50 | 0.4005 |
| PGT121 (Mammalian) | 1.716 | 3.744 |
| Nb VRC01-KDEL | >50 | 0.3337 |
| VRC07-523 (Mammalian) | 0.7221 | 4.152 |
| VRC07-523-LS(Mammalian) | 0.8532 | 4.498 |
| 830A (Mammalian) | 1.622 | 3.891 |
| LS HIG | 0.9101 | 4.482 |
| No ab | >50 | 0.3365 |

TABLE 12
FcγRIIB (CD32B)
EC50 >50 denotes samples where EC50 could not
be calculated due to flat curve.

| Antibody | EC50 (ug/mL) | AUC |
|---|---|---|
| Nb PGT121-LS | >50 | 0.2872 |
| Nb PGT121-YTE | >50 | 0.2555 |
| Nb PGT121 | >50 | 0.2776 |
| Nb PGT121-KDEL | >50 | 0.3997 |
| PGT121 (Mammalian) | 3.577 | 0.6357 |
| Nb VRC01-KDEL | >50 | 0.3550 |
| VRC07-523 (Mammalian) | ~2.095 | 1.263 |
| VRC07-523-LS(Mammalian) | ~1.945 | 1.359 |
| 830A (Mammalian) | 21.12 | 0.7820 |
| LS HIG | 13.63 | 0.8588 |
| No ab | >50 | 0.3711 |

TABLE 13
FcγRIII (CD16)
EC50 >50 denotes samples where EC50 could not
be calculated due to flat curve.

| Antibody | EC50 (ug/mL) | AUC |
|---|---|---|
| Nb PGT121-LS | >50 | 0.2447 |
| Nb PGT121-YTE | >50 | 0.2260 |
| Nb PGT121 | >50 | 0.2688 |
| Nb PGT121-KDEL | 3.572 | 0.9345 |
| PGT121 (Mammalian) | 1.047 | 1.516 |
| Nb VRC01-KDEL | >50 | 0.3697 |
| VRC07-523 (Mammalian) | 1.214 | 1.456 |
| VRC07-523-LS(Mammalian) | 0.6369 | 1.821 |
| 830A (Mammalian) | 1.459 | 1.499 |
| LS HIG | 0.6312 | 1.868 |
| No ab | >50 | 0.2328 |

### Example 3. Glycosylation Analysis

Glycosylation patterns were analyzed for the mammalian glycoprotein samples PGT121, 830A, and VRC07-523, and for the plant glycoproteins Nb VRC07-523-LS, Nb PGT121-LS and Nb VRC07-LS by nano liquid chromatography-nano electrospray ionization-tandem mass spectrometry (nLC-NSI-MS/MS). Glycosylation occurs on asparagine residues. Note that the asparagine numbering on each antibody are all equivalent to asparagine 297, the numbers are higher because the HIV antibodies have many amino acids comprising CDR₃.

Methods: 25 μg of each sample (i mg/mL; 50 μL/vial) was used for glycoproteomic analysis. The samples were reduced via DTT and alkylated via iodoacetamide before the proteins were enzymatically digested with trypsin at 37° C. overnight. After inactivation of the trypsin, the solutions were filtered. The peptides and glycopeptides were analyzed via nano-liquid chromatography nanospray ionization tandem mass spectrometry (nLC-NSI-MS/MS): The samples were injected to an Orbitrap Fusion Tribrid mass spectrometer through a nano-LC system and the glycopeptides were fragmented by HCD triggered CID program (based on glycoform oxonium ions). The data sets were processed by Byonic software and further analyzed by manual annotation.

For calculation of the relative percentages, the area under the curve of the most abundant peak (AUC^{most abundant Peak}) was calculated for each glycopeptide-derived mass. For each glycoform, the individual AUC^{most abundant peak} was divided through the sum of all AUC^{most abundant peak}.

### Results

### Glycopeptide Analysis

The mammalian glycoprotein samples PGT121, 830A, VRC07-523 and VRC07-523-L, and the plant glycoproteins Nb PGT121-LS and Nb VRC07-LS share overall very similar sequences, and the same sequence for the N-linked glycosylation motif in the Fc region (glycopeptide EEQYN*STYR).

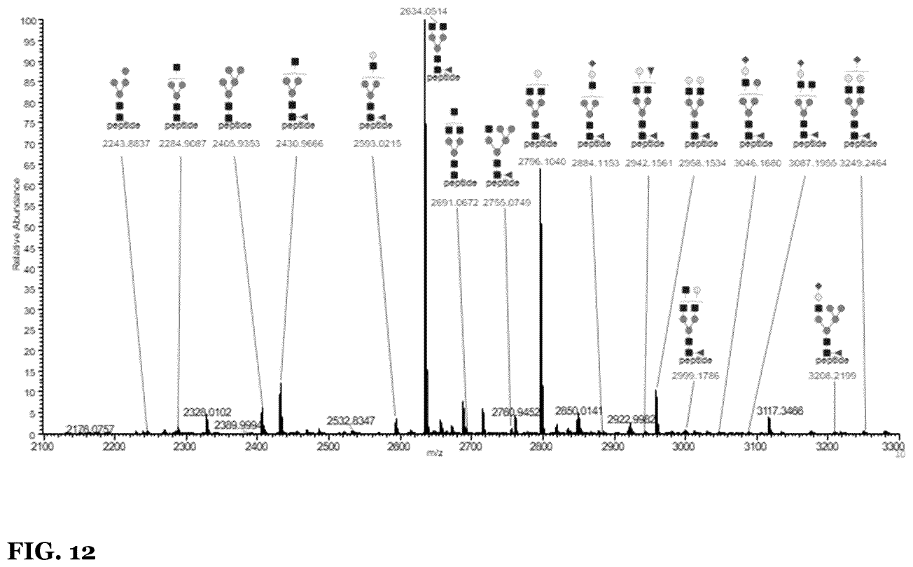

PGT121 sample (mammalian): The asparagine site NST on N-312 carried the N-glycosylation motif NST and was found to be fully glycosylated (99.98%). The analysis identified 17 glycoforms of which 96.48% were fucosylated. See FIG. 12 for spectra and glycoforms corresponding to each peak and Table 4 for the relative percentages of the identified glycoforms.

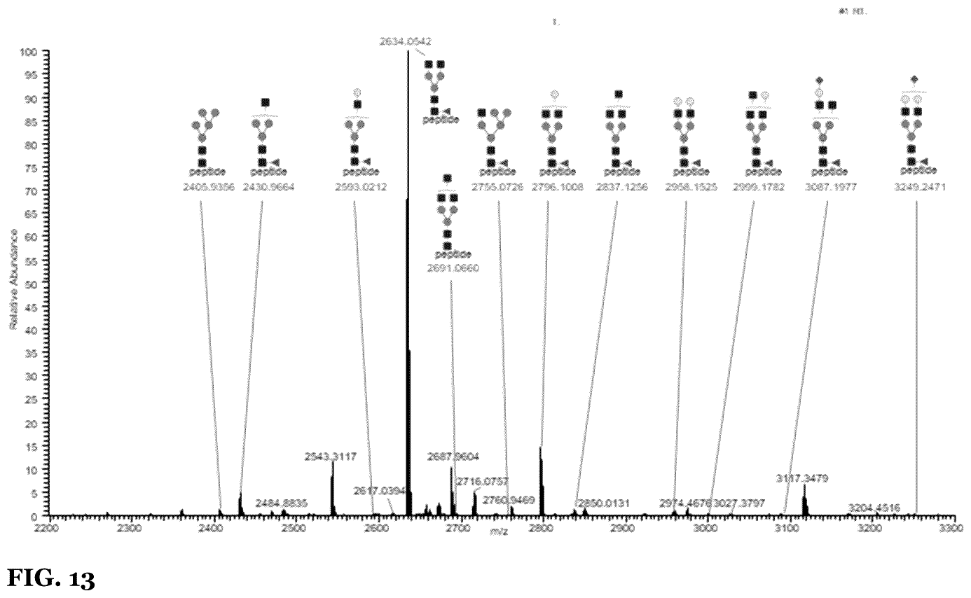

830A samples (mammalian): The asparagine site NST on N-328 carried the N-glycosylation motif NST and was found to be fully glycosylated (99.99%). The analysis identified 12 glycoforms of which 98.65% were fucosylated. See FIG. 13 for spectra and glycoforms corresponding to each peak and Table 4 for the relative percentages of the identified glycoforms.

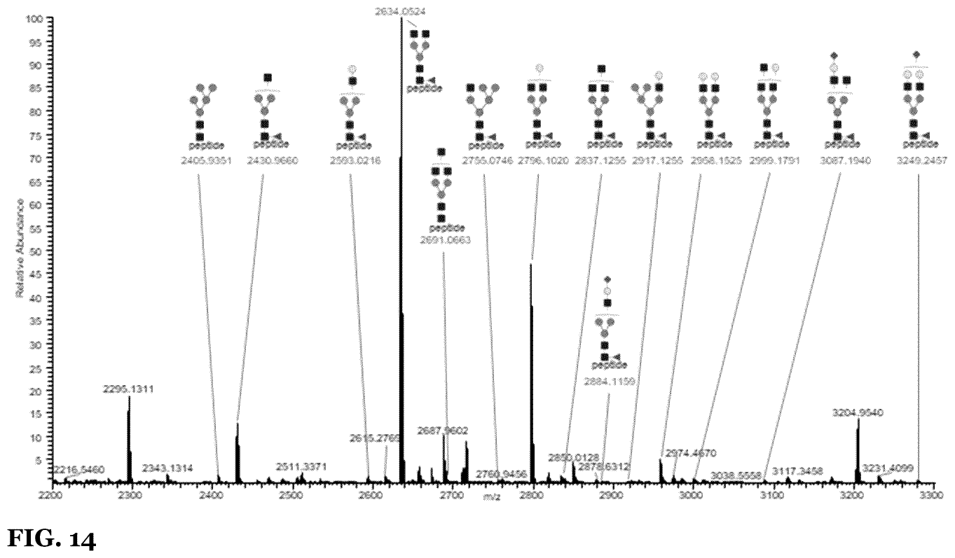

VRC07-523 sample (mammalian): The asparagine site NST on N-327 carried the N-glycosylation motif NST and was found to be fully glycosylated (99.98%). The analysis identified 14 glycoforms of which 98.71% were fucosylated. See FIG. 14 for spectra and glycoforms corresponding to each peak and Table 14 for the relative percentages of the identified glycoforms.

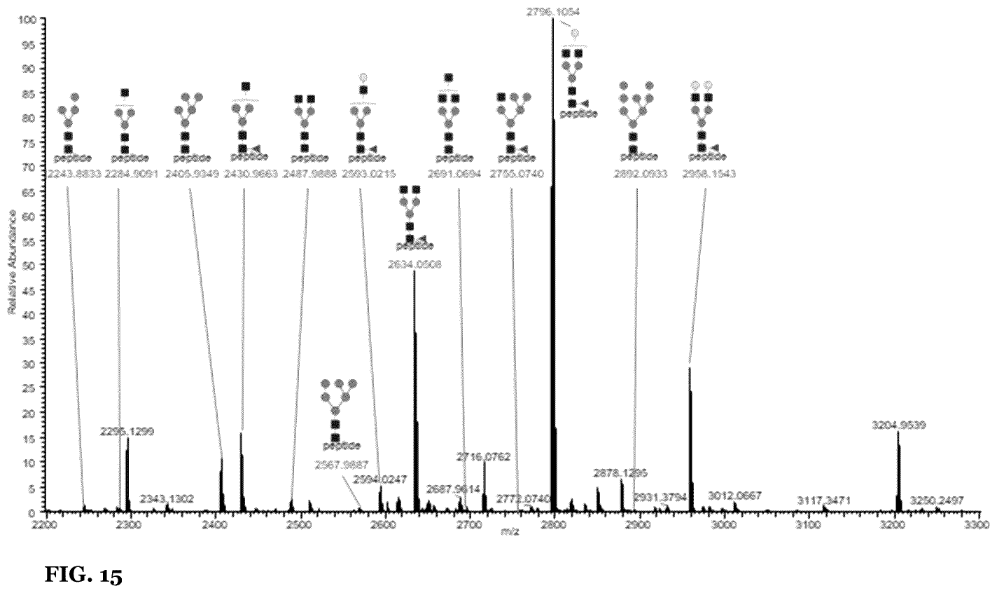

VRC07-523-LS sample (mammalian): The asparagine site NST on N-327 carried the N-glycosylation motif NST and was found to be fully glycosylated (99.98%). The analysis identified 13 glycoforms of which 92.34% were fucosylated. See FIG. 15 for spectra and glycoforms corresponding to each peak and Table 14 for the relative percentages of the identified glycoforms.

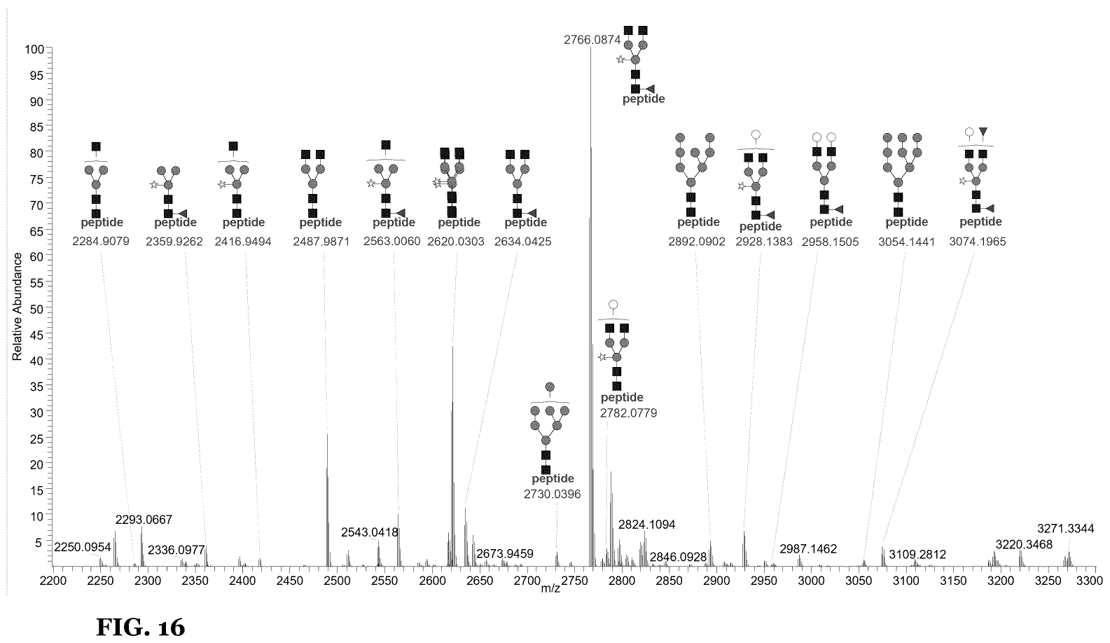

Nb PGT121-LS sample (plant): The asparagine site NST on N-312 carried the N-glycosylation motif NST and was present in both glycosylated (75.94%) and non-glycosylated forms (24.06%). The analysis identified 15 glycoforms of which 61.99% were fucosylated. See FIG. 16 for spectra and glycoforms corresponding to each peak and Table 4 for the relative percentages of the identified glycoforms.

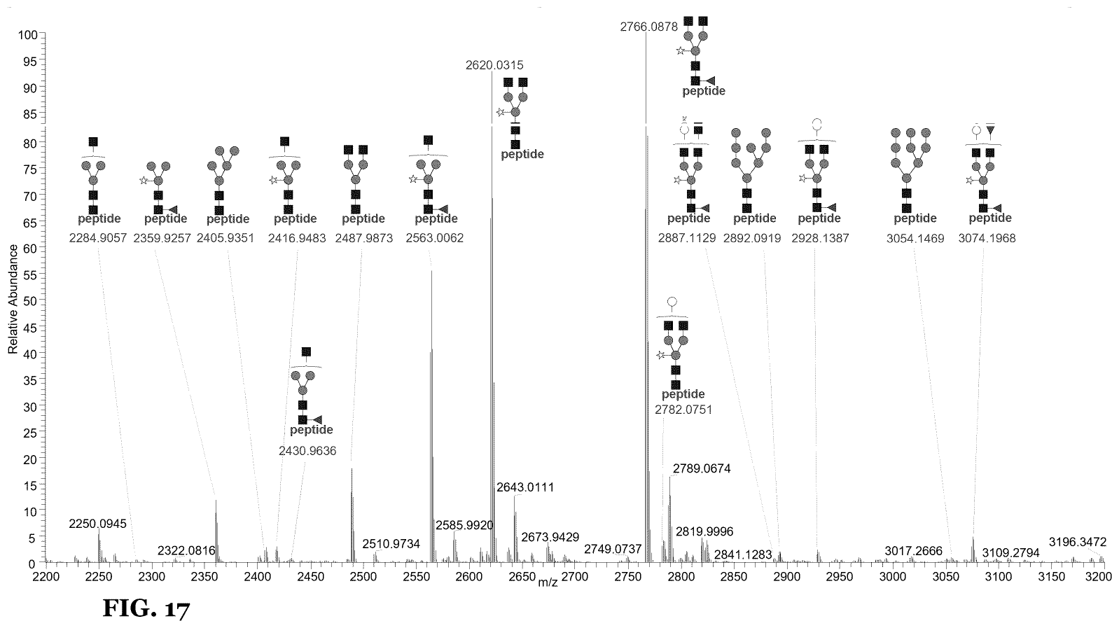

Nb VRC07-LS sample (plant): The asparagine site NST on N-312 carried the N-glycosylation motif NST and was in both glycosylated (73.95%) and non-glycosylated forms (26.05%). The analysis identified 15 glycoform of which 58.49% were fucosylated. See FIG. 17 for spectra and glycoforms corresponding to each peak and Table 4 for the relative percentages of the identified glycoforms.

Samples of mammalian PGT121, 830A, VRC07-523 and Nb VRC07-523-LS were identified as fully glycosylated (299.98% glycosylated) at peptide EEQYN*STYR. None of the glycoforms identified in these samples had xylose moieties. In contrast, samples of plant Nb PGT121-LS and Nb VRC07-LS had variable glycosylation at peptide EEQYN*STYR. For Nb PGT121-LS, eight of 15 glycoforms had β1,2-xylose moieties and for Nb VRC07-LS nine of 15 had β1,2-xylose moieties.

The structural assignments of the N-glycoforms is based on the molecular weight, composition analysis via nLC-MS/MS-derived data and follow the principles of the postulated biosynthetic pathway for N-glycoforms in mammalians or plants-depending on the sample origin as indicated.

### Example 4. Methionine Oxidation Analysis

Methionine oxidation was analysed on the glycoprotein samples PGT121, 830A, VRC07-523 and VRC07-523-LS by nano liquid chromatography-nano electrospray ionization-tandem mass spectrometry (nLC-NSI-MS/MS).

Methods: 25 μg of each sample (1 mg/mL; 50 μL/vial) was used for glycoproteomic analysis. The samples were reduced via DTT and alkylated via iodoacetamide before the proteins were enzymatically digested with trypsin at 37° C. overnight. After inactivation of the trypsin, the solutions were filtered. The peptides and glycopeptides were analyzed via nano-liquid chromatography nanospray ionization tandem mass spectrometry (nLC-NSI-MS/MS): The samples were injected to an Orbitrap Fusion Tribrid mass spectrometer through a nano-LC system and the glycopeptides were fragmented by HCD triggered CID program (based on glycoform oxonium ions). The data sets were processed by Byonic software and further analyzed by manual annotation. For calculation of the relative percentages, the area under the curve of the most abundant peak (AUC^{most abundant peak}) was calculated for each (glyco)peptide-derived mass. For each glycoform, the individual AUC^{most abundant peak} was divided through the sum of all AUC^{most abundant peak}.

Results: Analysis of the Methionine Residues and their Level of Oxidation (Forming Methionine Sulfoxide).

The samples PGT121, 830A, VRC07-523 and VRC07-523-LS were analyzed for methionine sulfoxide residues (oxidized methionine) based on the following sequences.

PGT121 comprises 4 peptides with 4 methionine residues which is shown in >PGT121 Heavy Chain (SEQ ID NO: i) and for completeness, the >PGT121 Light Chain (SEQ ID NO: 2) is shown:

(SEQ ID NO: 3)
QMQLQESGPGLVKPSETLSLTCSVSGASISDSYWSWIR;
(SEQ ID NO: 4)
YMDVWGNGTQVTVSSASTK
-peptide fully glycosylated; Man5
glycosylation chosen representatively;
(SEQ ID NO: 5)
DTLMISR;
and
(SEQ ID NO: 6)
WQQGNVFSCSVMHEALHNHYTQK

>PGT121 Heavy Chain (SEQ ID NO: 1)

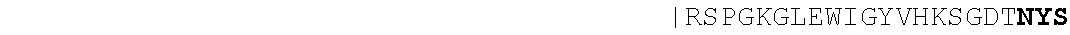

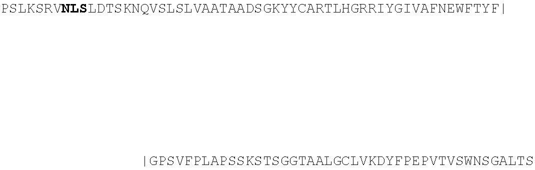

GVHTFPAVLQSSGLYSLSSVVTVPSSSLGTQTYICNVNHKPSNTKVDKKVEPKSCDKTHT

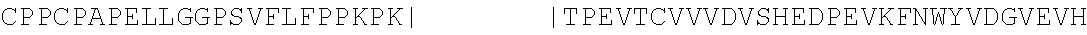

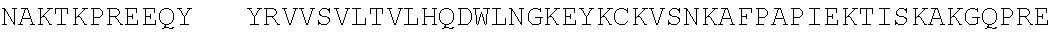

PQVYTLPPSRDELTKNQVSLTCLVKGFYPSDIAVEWESNGQPENNYKTTPPVLDSDGSFF

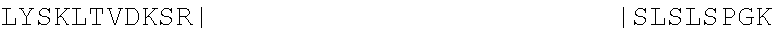

>PGT121 Light Chain (SEQ ID NO: 2)

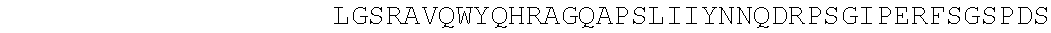

PFGTTATLTITSVEAGDEADYYCHIWDSRVPTKWVFGGGTTLTVLRTVAAPSVFIFPPSD
EQLKSGTASVVCLLNNFYPREAKVQWKVDNALQSGNSQESVTEQDSKDSTYSLSSTLTLS
KADYEKHKVYACEVTHQGLSSPVTKSENRGEC
signal peptide

830A comprises 1 peptide with 1 methionine residue which is shown in the >830A Heavy Chain (SEQ ID NO: 7) and for completeness, the >830A Light Chain (SEQ ID NO: 8) is shown: DTLMISR (SEQ ID NO: 9).

>830A Heavy Chain (SEQ ID NO: 7)

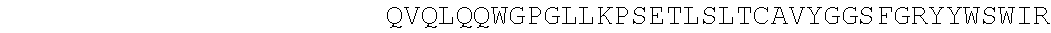

QSSGEGLEWLGQIDHTGSTTYNPSLKGRVTISIDSSTNQFSLKVTSVIAADTAVYYCARA
PSGYPGVSLYQYYGLDVWGQGTTVTVSPASTKGPSVFPLAPSSKSTSGGTAALGCLVKDY
FPEPVTVSWNSGALTSGVHTFPAVLQSSGLYSLSSVVTVPSSSLGTQTYICNVNHKPSNT

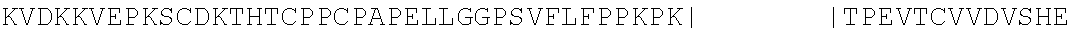

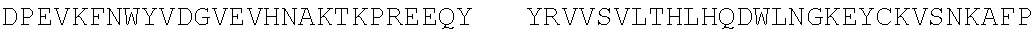

APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVLHEALHNHYTQKSLSLSPGK
>830A Light Chain (SEQ ID NO: 8)

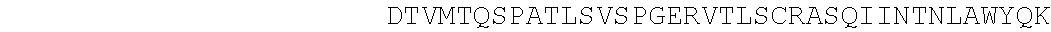

RPGQAPRLLIYAASARATGIPARFSGSGSGTEFTLTISGLQSEDSAVYYCQQYKHWPPYT
FGRGTKLEIRRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSENRGEC
signal peptid

VRC07-523 comprises 6 peptides with 7 methionine residues which are shown in the >VRC07-523 Heavy Chain (SEQ ID NO: 10) and for completeness, the >VRC07-523 Light Chain (SEQ ID NO: ii) is shown:

(SEQ ID NO: 12)
LSQSGGQMK;
(SEQ ID NO: 13)
KPGDSMRISCRASGYEFINCPINWIR;
(SEQ ID NO: 14)
RPEWMGWMK;
(SEQ ID NO: 15)
DMYSETAFLELR;
(SEQ ID NO: 16)
DTLMISR;
(SEQ ID NO: 17)
WQQGNVFSCSVMHEALHNHYTQK.

>VRC07-523 Heavy Chain (SEQ ID NO: 10)

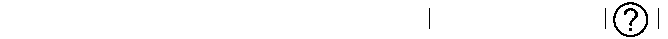

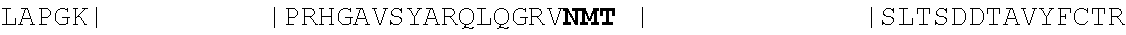

GKYCTARDYYNWDFEHWGQGTPVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTK

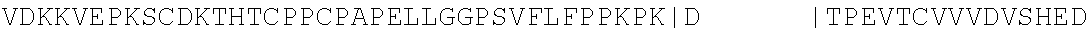

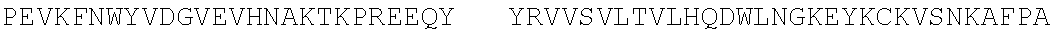

PIEKTISKAKGQPREPQVYTLPPSRDELTKNQVSLTCLVKGFYPSDIAVEWESNGQPENN

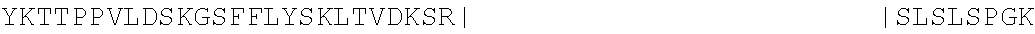

>VRC07-523 Light Chain (SEQ ID NO: 11)

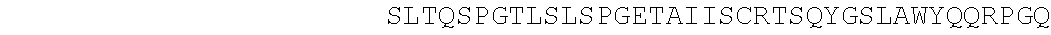

APRLVIYSGSTRAAGIPDRFSGSRWGPDYTLTISNLESGDFGVYYCQQYEFFGQGTKVQV
DIKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSENRGEC

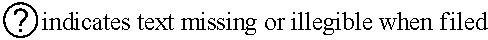

VRC07-523-LS comprises 5 peptides with 7 methionine residues which are shown in the >VRC07-523-LS Heavy Chain (SEQ ID NO: 18) and for completeness, the >VRC07-523-LS Light Chain (SEQ ID NO: 19) is shown:

(SEQ ID NO: 20)
LSQSGGQMK
(SEQ ID NO: 21)
KPGDSMRISCRASGYEFINCPINWIR
(SEQ ID NO: 22)
PEWMGWMKPRHGAVSYA
(SEQ ID NO: 23)
VTMTRDMYSETAFLE
(SEQ ID NO: 24)
DTLMISR

>VRC07-523-LS Heavy Chain (SEQ ID NO: 18)

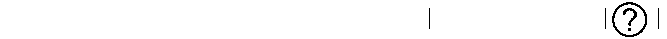

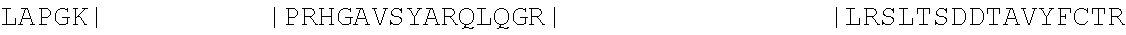

GKYCTARDYYNWDFEHWGQGTPVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTK

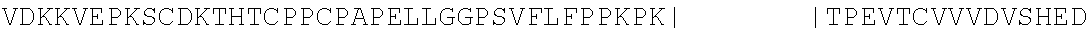

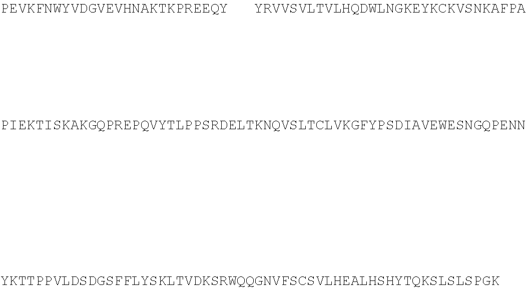

>VRC07-523-LS Light Chain (SEQ ID NO: 19)

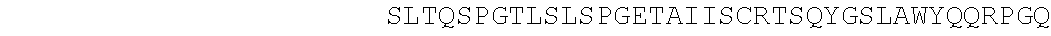

APRLVIYSGSTRAAGIPDRFSGSRWGPDYTLTISNLESGDFGVYYCQQYEFFGQGTKVQV
DIKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSENRGEC

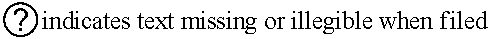

All of the antibodies show a high percentage (95.96-100%) of the native, non-oxidized form of methionine residues. See Table 15.

TABLE 15
Methionine oxidation on the glycoprotein samples
PGT121-LS, PGT121, 830A, VRC07-523 and VRC07-523-LS
Nb is Nicotiana benthamiana-derived. Mam is mammalian-derived.

| | Peptides | | | | | |
|---|---|---|---|---|---|---|
| | containing | | | | | |
| | methionines | | | | | |
| mAb Sample | detected | Percent Non-oxidized | | | | |
| Nb PGT121-LS | 3 | 96.24 | 100 | 94.98 | | |
| sample | | | | | | |
| Nb VRC07-523- | 3 | 94.41 | 92.33 | 96.3 | | |
| LS sample | | | | | | |
| Mam PGT121 | 4 | 97.69 | 92.74 | 98.66 | 99.61 | |
| sample | | | | | | |
| Mam VRC07-523 | 5 | 99.47 | 96.89 | 99.2 | 98.9 | 99.52 |
| sample | | | | | | |
| Mam VRC07-523- | 4 | 95.96 | 97.55 | 97.52 | 96.08 | |
| LS sample | | | | | | |
| Mam 830A | 1 | 98.72 | | | | |
| sample | | | | | | |

Four peptides containing 4 methionines were analysed for PGT121 and 6 peptides containing 7 methionines for VRC07-523; the DTMISR is conserved and is the only peptides analysed in both cases. All peptides are not always detected for analysis.
The LS mutation removes a methionine in each LS sample.

For the PGT121 sample (Table 15): The sequence carried 4 methionine residues on 4 peptides within the sequence of the heavy chain. 4 out of 4 methionine residues were covered by the method and analyzed. They were found to be almost fully present as methionine (98.66%—100%).

For the 830A sample (Table 15): The sequence carried 1 methionine residue within the sequence of the heavy chain, which was covered by MS analysis. It was found to be almost fully present as methionine (98.72%).

For the VRC07-523 sample (Table 15): The sequence carried 7 methionine residues on 6 peptides within the sequence of the heavy chain. 5 out of 7 methionine residues were covered by the method and analyzed. They were found to be almost fully present as methionine (96.89%—99.52%).

For the VRC07-523-LS sample (Table 15): The sequence carried 7 methionine residues on 6 peptides within the sequence of the heavy chain. 4 out of 7 methionine residues were covered by the method and analyzed. They were found to be almost fully present as methionine (95.96%—97.55%).

In proteins, oxidation of methionine residues to methionine sulfoxide can serve as an indicator to protein dysfunctionality and degradation. All samples from report YR112119Z (sent 02/13/2020) were identified with base level amounts of methionine sulfoxide (≤4.04%). “Base levels of oxidation are typically found at 0% to 5% in most proteins” (from Sen K I, Hepler R, Nanda H. Detection and Measurement of Methionine Oxidation in Proteins. Curr Protoc Protein Sci. 2017 Feb. 2; 87:14.16.1-14.16.11. doi: 10.1002/cpps.25).

The PTM assignment of methionine and methionine sulfoxide residues was based on the molecular weight of the peptides of interest and the analysis via nLC-MS/MS-derived data.

The results show that the plant antibodies were not oxidised. This indicates that an oxidised state was not responsible for the plant antibodies not crossing the placenta in Example 1, and that an oxidised state was not responsible for the plant antibodies failing to bind particular Fc receptors in Examples 2 and 3. As such, the data suggest that particular glycans are responsible for the plant antibodies not crossing the placenta and that one or more Fc receptors other than FcRN are responsible for antibodies crossing the placenta.

### REFERENCES

Simister N E et al., Eur J Immunol 26(7): 1527-1531, 1996)
Langel S N et al., PLoS Pathog 16(3): e1008303, 2020.
Rosenberg Y, Sack M, Montefiori D, Forthal D, Mao L Hernandez-Abanto S, et al. Rapid high-level production of functional HIV broadly neutralizing monoclonal antibodies in transient plant expression systems. PLoS One 2013; 8:e58724. doi: 10.1371/journal.pone.0058724 (inset in #0085)
Boesch A W, Miles A R, Chana Y N, Osei-Owusuc N Y, Ackerman M E. IgG Fc variant cross-reactivity between human and rhesus macaque FcgRs. MABS 2017; 9(3):455-465. doi: 10.1080/19420862.2016.1274845.
Chan Y N 1, Boesch A W, Osei-Owusu N Y, Emileh A, Crowley A R, Cocklin S L, et al. IgG Binding Characteristics of Rhesus Macaque FcγR. J Immunol. 2016; 197(7):2936-47. doi: 10.4049/jimmunol.1502252.

1. A method of treating a pregnant female in need thereof, wherein said pregnant female is administered a plant antibody or fragment thereof; wherein the plant antibody or fragment thereof is produced in a plant or plant cell to provide a plant antibody or fragment thereof that is unable to substantially cross the placenta if administered to a pregnant mother.

2. The method of claim 1, wherein the antibody if produced in a mammal or mammalian cell may cross the placenta.

3. The method of claim 1, wherein the plant antibody or fragment thereof is unable to substantially bind to FcγRI, FcγRIIA, FcγRIIB, FcγRIIIA or other Fc receptors.

4. The method of claim 1, wherein the plant antibody or fragment thereof is able to substantially bind to FcγRn.

5. The method of claim 1, wherein the plant antibody or fragment thereof comprises at least one plant specific glycan.

6. The method of claim 5, wherein the plant specific glycan comprises at least one sugar selected from the group consisting of: a fucose with a α1-3 linkage and a xylose with a β1,2 linkage.

7. The method of claim 5, wherein the plant specific glycan does not comprise galactose.

8. The method of claim 1, wherein the plant antibody or fragment thereof has a glycopeptide profile comprising at least one glycan selected from the group consisting of:
GlcNAc₂ Fuc Man₃ Xyl;
GlcNAc₂ Man₃ GlcNAc;
GlcNAc₂ Fuc Man₃ Xyl GlcNAc;
GlcNAc₂ Man₃ Xyl GlcNAc;
GlcNAc₂ Fuc Man₃ Xyl GlcNAc₂; and
GlcNAc₂ Fuc Man₃ Xyl GlcNAc₂ Hex.

9. The method of claim 8, wherein the glycopeptide profile comprises at least one glycan of a relative percentage selected from the group consisting of:
at least 2% of GlcNAc₂ Fuc Man₃ Xyl;
at least 2% of GlcNAc₂ Man₃ GlcNAc;
at least 2% of GlcNAc₂ Fuc Man₃ Xyl GlcNAc;
at least 2% of GlcNAc₂ Man₃ Xyl GlcNAc;
at least 2% of GlcNAc₂ Fuc Man₃ Xyl GlcNAc₂; and
at least 0.5% GlcNAc₂ Fuc Man₃ Xyl GlcNAc₂ Hex.

10. The method of claim 8, wherein the glycopeptide profile comprises at least one glycan of a relative percentage selected from the group consisting of:
2-10% of GlcNAc₂ Fuc Man₃ Xyl;
2-20% of GlcNAc₂ Man₃ GlcNAc;
2-30% of GlcNAc₂ Fuc Man₃ Xyl GlcNAc;
10-40% of GlcNAc₂ Man₃ Xyl GlcNAc;
20-60% of GlcNAc₂ Fuc Man₃ Xyl GlcNAc₂; and
0.5-6% GlcNAc₂ Fuc Man₃ Xyl GlcNAc₂ Hex.

11. The method of claim 9, wherein the glycan is selected from the group consisting of:
GlcNAc₂ Fuc Man₃ Xyl as shown in Error! Reference source not found.;
GlcNAc₂ Man₃ GlcNAc as shown in Error! Reference source not found.;
GlcNAc₂ Fuc Man₃ Xyl GlcNAc as shown in Error! Reference source not found.;
GlcNAc₂ Man₃ Xyl GlcNAc as shown in Error! Reference source not found.;
GlcNAc₂ Fuc Man₃ Xyl GlcNAc₂ as shown in Error! Reference source not found.; and
GlcNAc₂ Fuc Man₃ Xyl GlcNAc₂ Hex as shown in Error! Reference source not found.

12. The method of claim 1, wherein the plant antibody is selected from:
a) a human Fab and a human Fc;
b) a humanised Fab and a humanized Fcs from another animal;
c) a plant antibody or fragment thereof comprising a tag to target cellular compartments which modify glycans;
d) the plant antibody or fragment thereof of c), wherein the tag is a KDEL tag; and/or
e) an IgG1, IgG2, IgG3 or IgG4 isotype.

13. The method of claim 1, wherein the antibody produced in a mammal or mammalian cell is capable of causing damage to a foetus or newborn if administered to a pregnant mother.

14. The method of claim 1, wherein the plant or plant cell is selected from:
a) is a member of the Solanaceae family;
b) a tobacco plant;
c) a member of the Nicotiana genus;
d) Nicotiana benthamiana;
e) Nicotiana tabacum;
f) a plant cell transiently expressing the antibody; and/or
g) a plant comprising a polynucleotide integrated into the genome, wherein said polynucleotide encodes the antibody.

15. The method of claim 1, wherein the plant antibody or fragment thereof is substantially non-oxidised.

16. The method of claim 15, wherein the substantially non-oxidised plant antibody or fragment thereof comprises at least 95% methionine and 5% or less methionine sulfoxide.

17. The method of claim 1, where said plant antibody or fragment thereof is selected from any one of the antibodies listed in Table 1 or Table 2.

18. The method of claim 1, where said pregnant female is suffering from cancer, infectious disease, or an autoimmune disease.

19. The method of claim 18, wherein the cancer or condition is selected from the group consisting of an adrenal tumor, an acinar sarcoma, an astrocytoma, a bladder cancer, a bone cancer, a brain spinal cord cancer, a metastatic brain tumor, a breast cancer, a carotid body tumor, a cervical cancer, a chondrosarcoma, a spinal tumor, a kidney Chronic cell carcinoma, a clear cell carcinoma, a colon cancer, a colorectal cancer, fesmoplastic small round cell tumor, a fibroproliferative small round cell tumor, an extra-bone Myxoid chondrosarcoma, a fibrosarcoma, a fibrous dysplasia, a gallbladder or cholangiocarcinoma, a pregnancy trophoblastic disease, a germ cell tumor, a neck cancer, a hepatocellular carcinoma, an islet cell Tumor, Kaposi's sarcoma, a kidney cancer, a leukemia, a liposarcoma/malignant fat Adenoma, a liver cancer, a lymphoma, a lung cancer, a stromal cell tumor, a melanoma, a meningioma, a multiple endocrine tumor, a multiple myeloma, a myelodysplastic syndrome, a nerve A blastoma, a neuroendocrine tumor, an ovarian cancer, a pancreatic cancer, a papillary thyroid cancer, a parathyroid tumor, a peripheral schwannomas, a pituitary tumor, a prostate cancer, a posterior uveal melanoma, a primary central nervous system tumor, a renal metastasis cancer, a rhabdoid tumor, a rhabdomyosarcoma, a sarcoma, a skin cancer, a soft tissue sarcoma, a squamous cell carcinoma, a stomach cancer, a synovial sarcoma, a testicular cancer, a thymic carcinoma, a thymoma, a metastatic thyroid cancer, and a uterine cancer.

20. The method of claim 18, wherein the autoimmune disease or condition is selected from the group consisting of: type I diabetes mellitus (T1D), Crohn's disease, ulcerative colitis, myasthenia gravis, vitiligo, Graves' disease, Hashimoto's disease, Addison's disease, autoimmune gastritis, autoimmune hepatitis, rheumatoid disease, systemic lupus erythematosus, progressive systemic sclerosis and variants, polymyositis, dermatomyositis, primary biliary cirrhosis, autoimmune thrombocytopenia, Sjogren's syndrome, multiple sclerosis and psoriasis.


SEQUENCE LISTING (24 sequences)

SEQ ID NO: 1 - protein, 461 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 212 aa, synthetic construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 38 aa, Synthetic construct
QMQLQESGPGLVKPSETLSLTCSVSGASISDSYWSWIR

SEQ ID NO: 4 - protein, 19 aa, synthetic construct
YMDVWGNGTQVTVSSASTK

SEQ ID NO: 5 - protein, 7 aa, synthetic construct
DTLMISR

SEQ ID NO: 6 - protein, 23 aa, synthetic construct
WQQGNVFSCSVMHEALHNHYTQK

SEQ ID NO: 7 - protein, 478 aa, synthetic construct
MDMRVPAQLLGLLLLWLPGAKCQVQLQQWGPGLLKPSETLSLTCAVYGGSFGRYYWSWIRQSSGEGLEWLGQIDHTGSTTYNPSLKGRVTISIDSSTNQFSLKVTSVIAADTAVYYCARAPSGYPGVSLYQYYGLDVWGQGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 8 - protein, 237 aa, synthetic construct
MDMRVPAQLLGLLLLWLPGAKCDTVMTQSPATLSVSPGERVTLSCRASQIINTNLAWYQKRPGQAPRLLIYAASARATGIPARFSGSGSGTEFTLTISGLQSEDSAVYYCQQYKHWPPYTFGRGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 7 aa, synthetic construct
DTLMISR

SEQ ID NO: 10 - protein, 477 aa, synthetic construct
MDMRVPAQLLGLLLLWLPGAKCQVRLSQSGGQMKKPGDSMRISCRASGYEFINCPINWIRLAPGKRPEWMGWMKPRHGAVSYARQLQGRVNMTRDMYSETAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 230 aa, synthetic construct
MDMRVPAQLLGLLLLWLPGAKCSLTQSPGTLSLSPGETAIISCRTSQYGSLAWYQQRPGQAPRLVIYSGSTRAAGIPDRFSGSRWGPDYTLTISNLESGDFGVYYCQQYEFFGQGTKVQVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 9 aa, synthetic construct
LSQSGGQMK

SEQ ID NO: 13 - protein, 26 aa, synthetic construct
KPGDSMRISCRASGYEFINCPINWIR

SEQ ID NO: 14 - protein, 9 aa, synthetic construct
RPEWMGWMK

SEQ ID NO: 15 - protein, 12 aa, synthetic construct
DMYSETAFLELR

SEQ ID NO: 16 - protein, 7 aa, synthetic construct
DTLMISR

SEQ ID NO: 17 - protein, 23 aa, synthetic construct
WQQGNVFSCSVMHEALHNHYTQK

SEQ ID NO: 18 - protein, 477 aa, synthetic construct
MDMRVPAQLLGLLLLWLPGAKCQVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 19 - protein, 230 aa, synthetic construct; identical to SEQ ID NO: 11

SEQ ID NO: 20 - protein, 9 aa, synthetic construct
LSQSGGQMK

SEQ ID NO: 21 - protein, 26 aa, synthetic construct
KPGDSMRISCRASGYEFINCPINWIR

SEQ ID NO: 22 - protein, 17 aa, synthetic construct
PEWMGWMKPRHGAVSYA

SEQ ID NO: 23 - protein, 15 aa, synthetic construct
VTMTRDMYSETAFLE

SEQ ID NO: 24 - protein, 7 aa, synthetic construct
DTLMISR